(19) United States
(12) Patent Application Publication
(10) Pub. No.: US 2025/0170284 A1
(43) Pub. Date: May 29, 2025

(54) Hydroxamic Acid Macrocyclic Agents With Pendant Chelating Moieties And Complexes Thereof

(71) Applicant: LUMIPHORE, INC., Berkeley, CA (US)

(72) Inventors: David Tatum, Berkeley, CA (US); Darren Magda, San Leandro, CA (US); Nathaniel Butlin, Shawnigan Lake (CA)

(21) Appl. No.: 18/834,195

(22) PCT Filed: Feb. 1, 2023
(86) PCT No.: PCT/US2023/061735

§ 371 Date: Jul. 29, 2024

Related U.S. Application Data
(60) Provisional application No. 63/305,501, filed on Feb. 1, 2022.

(51) Int. Cl. A61K 51/04; C07D 401/14

(52) U.S. Cl. CPC A61K 51/0482; C07D 401/14

(57) ABSTRACT

The invention provides metal chelating agents and metal complexes of these agents of use in therapeutic and diagnostic applications.

### CROSS REFERENCE TO RELATED APPLICATIONS

The present disclosure claims priority to U.S. Provisional Patent Application No. 63/305,501 filed Feb. 1, 2022, which is hereby incorporated by reference.

### FIELD OF THE INVENTION

The invention relates to chelating agents and metal ion complexes thereof useful in therapeutic and diagnostic applications.

### BACKGROUND

Current radioimmunotherapy practice makes use of two classes of chelating agents: acyclic species based on diethylenetriamine pentaacetic acid (DTPA) or macrocyclic derivatives similar to 1,4,7,20-tetraazacyclododeccane N,N′,N″,N′″-tetraacetic acid (DOTA).

The former display more rapid association kinetics, while the DOTA-like compounds tend to produce a more stable complex, with the caveat that complexation typically requires harsher conditions such as high temperatures. A partial list of radiometals currently under clinical investigation (according to clinicaltrials.gov) includes actinium-225, bismuth-213, copper-64, gallium-67, gallium-68, holmium-166, indium-111, lutetium-177, rubidium-82, samarium-153, zirconium-89, strontium-89, technetium-99m, thorium-227, lead-212, and yttrium-90.

Lanthanide and actinide radiometal cations, in the absence of chelation, are largely deposited in bone, a significant concern given the potential for bone marrow suppression. The effective but nonselective amino-carboxylic acid ligands such as DTPA can deplete essential biological metal ions from patients, thus causing serious health problems. Selecting the correct type of chelating unit, therefore, is an important factor in achieving high selectivity toward the specific metal ion.

Toxicity concerns that have arisen recently following the use of MRI contrast agents such as Gd⁺³ DTPA, clearly underscore the insufficient control of the metal cation biodistribution by this chelating group. Similarly, radiometal loss can lead to a loss of signal specificity by targeted radiodiagnostics. Therefore, there is a recognized, compelling need for improved chelating agents for use in radioimmunotherapy. Such chelating agents and complexes and methods of their use are provided by the present invention.

### SUMMARY OF INVENTION

The present invention provides a new class of metal chelating agents and metal ion chelates of these agents which are particularly useful in therapeutic and/or diagnostic applications.

The chelating agents of the invention have numerous favorable properties, which are not generally available, either alone or in combination, in macrocyclic chelating agents. For instance, exemplary compounds of the invention metallate rapidly. Many metals hydrolyze at, or above physiological pH, forming metal-hydroxide complexes that cannot be chelated readily. In contrast, exemplary chelating agents of this disclosure have low pKa, such that they are anionic at neutral pH. This allows rapid metalation under conditions (e.g., pH 5) where metal ions (particularly tetravalent, but even trivalent) are less likely to hydrolyze.

Counterintuitively, in general, metal chelates of the chelating agents according to this disclosure are highly thermodynamically stable despite the ability of the chelating agents to rapidly metallate. Chelates according to this disclosure exhibit high stability with +3 and +4 metals in physiologically relevant conditions.

Exemplary metal chelates of the chelating agents of the invention are also resistant to oxidation by O₂, ubiquitous in the atmosphere and in living organisms.

The chelating agent/metal ion chelate of this invention comprise a mixture of bridging chelating moieties and pendant chelating moieties that are linked together via a scaffold moiety comprising the bridging chelating moieties to have a structure according to Formula A:

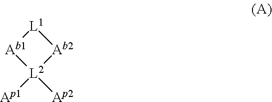

wherein L¹ and L² are independently selected scaffold moieties; A^{b1} and A^{b2} are independently selected bridging chelating moieties; and A^{p1} and A^{p2} are independently selected pendant chelating moieties.

The bridging chelating moieties of the present invention are independently selected from:

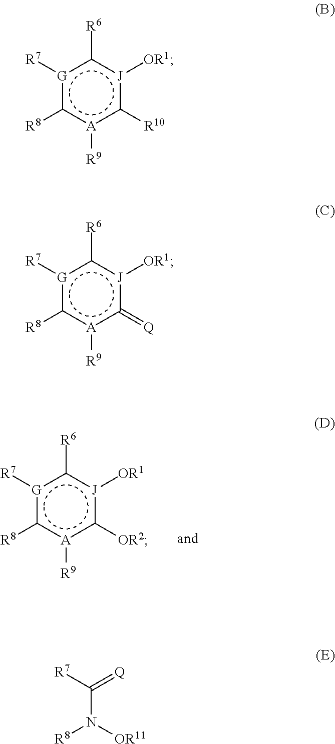

wherein A and G are independently selected from carbon, nitrogen and oxygen. J is selected from carbon and nitrogen. Q is 0, S or SH. Each R¹ and R² is independently selected from H, an enzymatically labile group, a hydrolytically labile group, a metabolically labile group, a photolytically labile group and a single negative charge. Each R⁶, R⁷, R⁸, R⁹, and R¹⁰ is independently selected from a bond to L¹ or L², alkanediyl attached to L¹ or L², H, substituted or unsubstituted alkyl, substituted or unsubstituted heteroalkyl, halogen, CN, —CF₃, —C(O)R¹⁷, —SO₂NR¹⁷R¹⁸, —NR¹⁷R¹⁸, —OR¹⁷, —S(O)₂R¹⁷, —COOR¹⁷, —S(O)₂OR¹⁷, —OC(O)R¹⁷, —C(O)NR¹⁷R¹⁸, —NR¹⁷C(O)R¹⁸, —NR¹⁷SO₂R¹⁸, and —NO₂, wherein at least two of R⁶, R⁷, R⁸, R⁹, and R¹⁰ are optionally joined to form a ring system selected from substituted or unsubstituted cycloalkyl, substituted or unsubstituted heterocycloalkyl, substituted or unsubstituted aryl and substituted or unsubstituted heteroaryl. R¹⁷ and R¹⁸ are independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl; and R¹⁷ and R¹⁸, together with the atoms to which they are attached, are optionally joined to form a 5-, 6- or 7-membered ring. When A is oxygen, R⁹ is not present; and when G is oxygen, R⁷ is not present. A^{b1} and A^{b2} are attached to L¹ and L² through two members selected from R⁶, R⁷, R⁸, R⁹ and R¹⁰; and A^{p1} and A^{p2} are attached to L² through a member selected from R⁶, R⁷, R⁸, R⁹ and R¹⁰.

In an exemplary embodiment, at least one of the pendent chelating moieties is (E). In various embodiments, both pendent chelating moieties are (E).

In various embodiments, one or more of R⁶, R⁷, R⁸, R⁹ and R¹⁰ is a linker to a reactive functional group or to a targeting moiety. In these compounds the atom to which this linker is attached is not involved in a group bridging L¹ and L². In various embodiments, the linker is substituted or unsubstituted alkyl or substituted or unsubstituted heteroalkyl. An exemplary reactive functional group to which the linker is attached is an amine, an alcohol, a carboxylic acid, or a reactive derivative thereof (e.g., maleimide, isocyanate, isothiocyanate), ester (e.g., active ester), or other reactive functional groups set forth herein. In exemplary embodiments, the reactive functional group is a halide (e.g., acyl halide).

In the structures of the bridging chelating moieties above, the dashed circle indicates that the ring system in which it is found includes 0, 1, 2, or 3 degrees of unsaturation and is selected from aromatic and non-aromatic ring systems.

Exemplary chelating agent/metal ion chelate of the present invention also comprise a linker to a reactive functional group or a linker to a targeting moiety, therefore, the chelating agent/metal ion chelate provided herein can be directed to a site of interest for therapeutic or diagnostic purposes. This linker, or these linkers, can be attached to any point on the molecule, e.g., at a nitrogen atom or a carbon atom of the scaffold, one or more bridging chelating moiety, or one or more pendant chelating moiety.

Exemplary chelating agent/metal ion chelate of the invention include compounds of the structure according to Formula I.

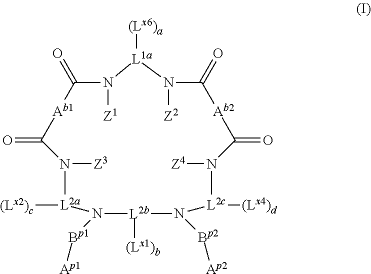

wherein L^{x1}, L^{x2}, L^{x3}, L^{x4}, L^{x6}, Z¹, Z², Z³ and Z⁴ are each independently selected from H, substituted or unsubstituted alkyl and substituted or unsubstituted heteroalkyl, or a linker to a reactive functional group or a targeting moiety. An exemplary linker is selected from a C₁-C₂₀ substituted or unsubstituted alkyl moiety, and a 1-20 atom length substituted or unsubstituted heteroalkyl moiety; L^{1a}, L^{2a}, L^{2b}, and L^{2c} are independently selected substituted or unsubstituted C₂-C₆ alkyl; each index a, b, c, or d is independently selected from 0 and 1; A^{b1} and A^{b2} are independently selected from: (i) substituted or unsubstituted, saturated or unsaturated alkyl selected from straight-chain, branched chain, and cycloalkyl; (ii) substituted or unsubstituted, saturated or unsaturated heterocycloalkyl; and (iii) substituted or unsubstituted heteroaryl, each of (i), (ii) and (iii) comprising an oxo and a NOH moiety; B^{p1} and B^{p2} are branching groups independently selected from: a bond, C1-C₃ alkyl comprising an oxo group, and —C(O)—; A^{p1} and A^{p2} are independently selected from: (a) C₁-C₆ alkyl substituted with a member selected from: (i) substituted and unsubstituted, saturated and unsaturated cycloalkyl; (ii) substituted and unsubstituted, saturated and unsaturated heterocycloalkyl, each comprising an oxo and a NOH moiety; and (b) (CH₂)ₓC(O)NR¹NR²R³, (CH₂)ₓN(OH)C(O)R¹ and (CH₂)ₓC(O)N(OH)R¹, wherein R¹, R², and R³ are independently selected from H and C₁-C₆ substituted and unsubstituted alkyl; and x is integer selected from 1, 2 and 3. Z¹, Z², Z³ and Z⁴ are independently selected from H, substituted or unsubstituted alkyl and substituted or unsubstituted heteroalkyl.

In an exemplary embodiment, one or more of Z¹, Z², Z³ and Z⁴ is an independently selected linker as that term is defined herein. In various embodiments, one or more of Z¹, Z², Z³ and Z⁴ is an independently selected linker to a reactive functional group or to a targeting moiety.

In an exemplary embodiment, each a, b, c, and d is 0, or each L^{x1}, L^{x2}, L^{x4} and L^{x6} is H, Z¹-Z⁴ are each H, and the chelating agent/metal ion chelate does not have a linker to a reactive functional group or a linker to a targeting moiety. In some embodiments, one or more of the index a, b, c, or d is 1, at least one L^{x6} is other than H, and the chelating agent/metal ion chelate includes one or more linker to a reactive functional group or a linker to a targeting moiety. In some embodiments, each a, b, c, and d is 0, or each L^{x1}, L^{x2}, L^{x4} and L^{x6} is H, and one or more of Z¹-Z⁴ is a linker to a reactive functional group or a linker to a targeting moiety.

Exemplary advantages of the compounds of the present invention are that such chelating agent/metal ion chelates bind the metal ion isotope rapidly, so that the chelators are compatible with the practicalities of clinical laboratory preparation. These compounds also bind the metal cation stably so that the release of the metal ion isotope prior to its decay is minimized in vivo. The solution to both of these opposing design challenges are embodied by the pre-organized chelating groups that retain a sufficient degree of flexibility, as described in the present invention.

Compounds of the present invention and metal ion complexes thereof are particularly useful for targeted radioisotope applications and sensitized luminescence applications (such as Eu sensitized luminescence immunoassays).

### DESCRIPTION OF EMBODIMENTS

### Definitions

Where substituent groups are specified by their conventional chemical formulae, written from left to right, they optionally equally encompass the chemically identical substituents, which would result from writing the structure from right to left, e.g., —CH₂O— is intended to also recite —OCH₂—.

The term “alkyl”, by itself or as part of another substituent, means a straight or branched chain hydrocarbon, which may be fully saturated, mono- or polyunsaturated and includes mono-, di- and multivalent radicals. Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds (i.e., alkenyl and alkynyl moieties). Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl” can refer to “alkylene”, which by itself or as part of another substituent means a divalent radical derived from an alkane, as exemplified, but not limited, by —CH₂CH₂CH₂CH₂—. Typically, an alkyl (or alkylene) group will have from 1 to 30 carbon atoms. A “lower alkyl” or “lower alkylene” is a shorter chain alkyl or alkylene group, generally having eight or fewer carbon atoms. In some embodiments, alkyl refers to an alkyl or combination of alkyls selected from C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉ and C₃₀ alkyl. In some embodiments, alkyl refers to C₁-C₂₅ alkyl. In some embodiments, alkyl refers to C₁-C₂₀ alkyl. In some embodiments, alkyl refers to C₁-C₁₅ alkyl. In some embodiments, alkyl refers to C₁-C₁₀ alkyl. In some embodiments, alkyl refers to C₁-C₆ alkyl.

The term “heteroalkyl,” by itself or in combination with another term, means an alkyl in which one or more carbons are replaced with one or more heteroatoms selected from the group consisting of O, N, Si and S, (preferably O, N and S), wherein the nitrogen and sulfur atoms may optionally be oxidized and the nitrogen heteroatom may optionally be quaternized. The heteroatoms O, N, Si and S may be placed at any interior position of the heteroalkyl group or at the position at which the alkyl group is attached to the remainder of the molecule. In some embodiments, depending on whether a heteroatom terminates a chain or is in an interior position, the heteroatom may be bonded to one or more H or substituents such as (C₁, C₂, C₃, C₄, C₅ or C₆) alkyl according to the valence of the heteroatom. Examples of heteroalkyl groups include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, and —CH═CH—N(CH₃)—CH₃. No more than two heteroatoms may be consecutive, as in, for example, —CH₂—NH—OCH₃ and —CH₂—O—Si(CH₃)₃, and in some instances, this may place a limit on the number of heteroatom substitutions. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. The designated number of carbons in heteroforms of alkyl, alkenyl and alkynyl includes the heteroatom count. For example, a (C₁, C₂, C₃, C₄, C₅ or C₆) heteroalkyl will contain, respectively, 1, 2, 3, 4, 5 or 6 atoms selected from C, N, O, Si and S such that the heteroalkyl contains at least one C atom and at least one heteroatom, for example 1-5 C and 1 N or 1-4 C and 2 N. Further, a heteroalkyl may also contain one or more carbonyl groups. In some embodiments, a heteroalkyl is any C₂-C₃₀ alkyl, C₂-C₂₅ alkyl, C₂-C₂₀ alkyl, C₂-C₁₅ alkyl, C₂-C₁₀ alkyl or C₂-C₆ alkyl in any of which one or more carbons are replaced by one or more heteroatoms selected from O, N, Si and S (or from O, N and S). In some embodiments, each of 1, 2, 3, 4 or 5 carbons is replaced with a heteroatom. The terms “alkoxy,” “alkylamino” and “alkylthio” (or thioalkoxy) are used in their conventional sense, and refer to those alkyl and heteroalkyl groups attached to the remainder of the molecule via an oxygen atom, a nitrogen atom (e.g., an amine group), or a sulfur atom, respectively.

The terms “cycloalkyl” and “heterocycloalkyl”, by themselves or in combination with other terms, refer to cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The term “aryl” means a polyunsaturated, aromatic substituent that can be a single ring or optionally multiple rings (preferably 1, 2 or 3 rings) that are fused together or linked covalently. In some embodiments, aryl is a 3, 4, 5, 6, 7 or 8 membered ring, which is optionally fused to one or two other 3, 4, 5, 6, 7 or 8 membered rings. The term “heteroaryl” refers to aryl groups (or rings) that contain 1, 2, 3 or 4 heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl.

In some embodiments, any of alkyl, heteroalkyl, cycloalkyl, heterocycloalkyl, aryl and heteroaryl is optionally substituted. That is, in some embodiments, any of these groups is substituted or unsubstituted. In some embodiments, substituents for each type of radical are selected from those provided below.

Substituents for the alkyl, heteroalkyl, cycloalkyl and heterocycloalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents”. In some embodiments, an alkyl group substituent is selected from -halogen, —OR′, =O, =NR′, =N—OR′, —NR′R″, —SR′, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)=NR″″, —NR—C(NR′R″)=NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. In one embodiment, R′, R″, R′″ and R″″ are each independently selected from hydrogen, alkyl (e.g., C₁, C₂, C₃, C₄, C₅ and C₆ alkyl). In one embodiment, R′, R″, R′″ and R″″ each independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. In one embodiment, R′, R″, R′″ and R″″ are each independently selected from hydrogen, alkyl, heteroalkyl, cycloalkyl, heterocycloalkyl, aryl, heteroaryl, alkoxy, thioalkoxy groups, and arylalkyl. When R′ and R″ are attached to the same nitrogen atom, they can be combined with the nitrogen atom to form a 5-, 6-, or 7-membered ring. For example, —NR′R″ can include 1-pyrrolidinyl and 4-morpholinyl. In some embodiments, an alkyl group substituent is selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl.

Similar to the substituents described for the alkyl radical, substituents for the aryl and heteroaryl groups are generically referred to as “aryl group substituents”. In some embodiments, an aryl group substituent is selected from -halogen, —OR′, =O, =NR′, =N—OR′, —NR′R″, —SR′, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′)=NR″″, —NR—C(NR′R″)=NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system. In some embodiments, R′, R″, R′″ and R″″ are independently selected from hydrogen and alkyl (e.g., C₁, C₂, C₃, C₄, C₅ and C₆ alkyl). In some embodiments, R′, R″, R′″ and R″″ are independently selected from hydrogen, substituted or unsubstituted alkyl, substituted or unsubstituted heteroalkyl, substituted or unsubstituted aryl and substituted or unsubstituted heteroaryl. In some embodiments, R′, R″, R′″ and R″″ are independently selected from hydrogen, alkyl, heteroalkyl, aryl and heteroaryl. In some embodiments, an aryl group substituent is selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl.

Two of the substituents on adjacent atoms of the aryl or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

The term “acyl” refers to a species that includes the moiety —C(O)R, where R has the meaning defined herein. Exemplary species for R include H, halogen, substituted or unsubstituted alkyl, substituted or unsubstituted aryl, substituted or unsubstituted heteroaryl, and substituted or unsubstituted heterocycloalkyl. In some embodiments, R is selected from H and (C₁-C₆)alkyl.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like. In some embodiments, halogen refers to an atom selected from F, Cl and Br.

The term “heteroatom” includes oxygen (O), nitrogen (N), sulfur (S) and silicon (Si). In some embodiments, a heteroatom is selected from N and S. In some embodiments, the heteroatom is O.

Unless otherwise specified, the symbol “R” is a general abbreviation that represents a substituent group that is selected from acyl, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound includes more than one R, R′, R″, R′″ and R″″ group, they are each independently selected.

For groups with solvent exchangeable protons, the ionized form is equally contemplated. For example, —COOH also refers to —COO⁻ and —OH also refers to —O⁻.

Any of the compounds disclosed herein can be made into a pharmaceutically acceptable salt. The term “pharmaceutically acceptable salts” includes salts of compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., Journal of Pharmaceutical Science, 66: 1-19 (1977)). Certain specific compounds of the present invention contain both basic and acidic functionalities that allow the compounds to be converted into either base or acid addition salts. The neutral forms of the compounds are preferably regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides any of the compounds disclosed herein in a prodrug form. Prodrugs of the compounds described herein are those compounds that readily undergo chemical changes under physiological conditions to provide the compounds of the present invention.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

The compounds of the present invention may also contain unnatural proportions of atomic isotopes at one or more of the atoms that constitute such compounds. For example, the compounds may be labeled with deuterium (²H) or radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

The symbol [symbol] , displayed perpendicular to a bond (or other moiety), indicates the point at which the displayed bond or moiety is attached to the remainder of the molecule.

Where numeric ranges are specified, e.g., C₁-C₂₀, or 1-20, the ranges are understood to encompass all values intermediate to the endpoints of the range and all combinations of two intermediate values or one intermediate value and one endpoint. Thus, C₁-C₂₀ encompasses, e.g., C₁-C₁₈, C₁₈-C₂₀, and C₁₀-C₁₈. In some embodiments, the span between the starting value and ending value of a range is 2, 3, or 4 of the relevant units, e.g. C₂-C₄, C₂-C₅ or C₂-C₆.

In some embodiments, the definition of terms used herein is according to IUPAC.

### Compositions

The invention provides a novel genus of chelating agents/metal ion chelates and metal complexes thereof. Generally, chelating agent/metal ion chelate of the invention comprise a plurality of bridging and pendent chelating moieties that are linked together by way of a scaffold moiety.

Chelating agents/metal ion chelates of the present invention are particularly useful for targeted radioisotope applications and sensitized luminescence applications (such as Eu sensitized luminescence immunoassays). Exemplary chelating agents/metal ion chelates of the present invention stably coordinate metal cations and display facile chelation kinetics, and robust chelation thermodynamics. Additionally, the introduction of cyclic or acyclic hydroxamate moieties (in place of hydroxypyridinone moieties previously disclosed) provides chelators with higher pKa values than those of exemplary all HOPO chelating agents/metal ion chelates, resulting in greater thermodynamic stability constants with exemplary metal ions. The flexibility of the linear hydroxamates reduces kinetic stability of the metal ion chelates, effectively improving the rates of metal ion chelation and metal ion exchange, both of which are important practical factors for quantitative radiolabeling. The cyclic hydroxamate chelators described here are preorganized for metal ion chelation, thereby engendering additional stability improvements over the acyclic hydroxamate analogs. The cyclic hydroxamates also have the advantage of allowing tuning of the size of the bidentate bite angle (i.e. changing the distance between two donor atoms of the same moiety) by modulating the number of methylene units within the ring, which may broaden the range of metal ion sizes that these chelators can accommodate. Similarly, the thiohydroxamate chelating agents disclosed herein broaden the scope of the genus of chelating agents to include a subgenus useful for chelating softer metals ions (e.g. Pb²⁺ and Cu²⁺). Exemplary acyclic hydroxamate containing chelators of this disclosure improve the solubility of the metal ion chelates, which may be important for in vivo applications. Exemplary cyclic and acyclic hydroxamates also avoid deleterious sequestration by siderocalin in vivo, which has been demonstrated to bind hydroxypyridinone-containing metal ion chelates in the literature (e.g. Carter K, Deblonde G, Lohrey T, Rupert P, Allaire M, An D, Strong R, Abergel, R. Acta Cryst, 2019, A75, a73). Additionally, the use of hydroxamate chelators in the pendant positions of exemplary chelating agents/metal ion chelates provides materials with larger Eu(III) quantum yields, as the replaced pendant hydroxypyridinones have been shown to be less efficient (20-25%) at sensitizing Eu(III) luminescence than the bridging chromophores (30-37%).

An exemplary chelating agent of the invention is a macrocycle comprising a plurality of internal and pendent chelating moieties. Particularly useful chelating agents contain a number of chelating moieties sufficient to provide, for example, at least 4, 6, 8 or 10 heteroatoms such as oxygen that coordinate with a metal ion to form a metal ion chelate. The heteroatoms such as oxygen, and sulfur provide electron density for forming coordinate bonds with a positively charged metal ion, and such heteroatoms can thus be considered “donors”. In some embodiments, the plurality of chelating moieties of a chelating agent comprises a plurality of oxygen donors, and a metal ion (such as a radionuclide) is chelated to the chelating agent via at least one of the oxygen donors. In some embodiments, a chelating agent comprises a plurality of oxygen donors and a metal ion (such as a radionuclide) is chelated to the ligand via a plurality of or all of the oxygen donors.

### Chelating Agents

In one aspect, the invention provides a chelating agent having the structure:

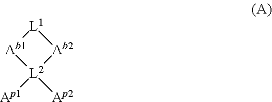

wherein L¹ and L² are independently selected scaffold moieties; A^{b1} and A^{b2} are independently selected bridging chelating moieties; and A^{p1} and A^{p2} are independently selected pendant chelating moieties. Scaffold moieties, bridging chelating moieties and pendant chelating moieties are as defined herein.

Exemplary scaffold moieties include substituted and unsubstituted alkyl, substituted and unsubstituted heteroalkyl, substituted and unsubstituted aryl, and substituted and unsubstituted heteroaryl moieties. Bridging and pendant chelating moieties are described in more detail hereinbelow.

In some embodiments, the chelating agent/metal ion chelate comprises a linker to a reactive functional group, or a linker to a targeting moiety. In some embodiments, at least any one or more of L¹, L², A^{b1}, A^{b2}, A^{p1} or A^{p2} is substituted with a linker to a reactive functional group, or a linker to a targeting moiety. Exemplary linkers to a reactive functional group and linkers to a targeting moiety are as defined herein. In some embodiments, the reactive functional group is a protected reactive functional group.

Any of the combinations of L¹, L², A^{b1}, A^{b2}, A^{p1}, and A^{p2}, as these groups are described herein, are encompassed by this disclosure and are expressly provided by the invention.

In some embodiments, the chelating agent/metal ion chelate comprises one or more modifying moieties. The one or more modifying moieties can be the same or different.

In some embodiments, A^{b1} and A^{b2} are each independently selected from:
(i) substituted and unsubstituted, saturated and unsaturated alkyl selected from straight-chain, branched chain, and cycloalkyl;
(ii) substituted and unsubstituted, saturated and unsaturated heterocycloalkyl; and
(iii) substituted and unsubstituted heteroaryl, wherein each of (i), (ii) and (iii) comprises an oxo and a NOH moiety.

In some embodiments, A^{b1} and A^{b2} are each independently selected from:

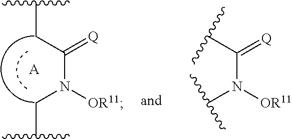

in which ring A is a four-, five-, or six-member ring, and has at least one degree of unsaturation or is saturated; and Q is independently selected from O, S, and SH. The dashed curve in ring A denotes optional unsaturation, i.e., the ring may be saturated or include 1, or 2 degrees of unsaturation at any position appropriate to satisfy the rules of valency. R″ is selected from H, an enzymatically labile group, a hydrolytically labile group, a metabolically labile group, a photolytically labile group and a single negative charge. As will be noted by those of skill in the art, the formulae above allow for the attachment of the chelating moiety to the remainder of the chelating agent in either an “up” or a “down” orientation (i.e. C=O can be in the superior or inferior position with respect to NOR¹¹). This representation is intentional and chelating agent/metal ion chelate containing chelating moieties in either configuration or both configurations are encompassed by the instant invention.

In various embodiments, ring A is aromatic.

In an exemplary embodiment, A^{b1} and A^{b2}, and A^{p1} and A^{p2} are each independently selected from:

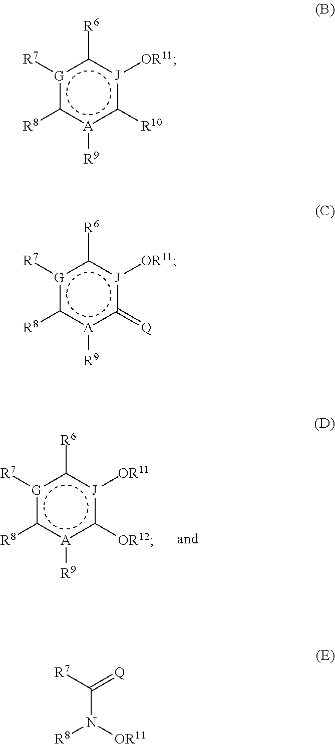

in which A and G are independently selected from C, N and O; J is selected from C and N; and Q is selected from 0, S, and SH. Each R¹⁰ and R″ is independently selected from H, an enzymatically labile group, a hydrolytically labile group, a metabolically labile group, a photolytically labile group and a single negative charge. Each R⁶, R⁷, R⁸, R⁹ is independently selected from a bond to L¹ or L², substituted or unsubstituted alkanediyl attached to and joining L¹ and L², H, substituted or unsubstituted alkyl, substituted or unsubstituted heteroalkyl, halogen, CN, —CF₃, —C(O)R¹⁷, —SO₂NR¹⁷R¹⁸, —NR¹⁷R¹⁸, —OR¹⁷, —S(O)₂R¹⁷, —COOR¹⁷, —S(O)₂OR¹⁷, —OC(O)R¹⁷, —C(O)NR¹⁷R¹⁸, —NR¹⁷C(O)R¹⁸, —NR¹⁷SO₂R″, and —NO₂, wherein at least two of R⁶, R⁷, R⁸, R⁹, and R¹⁰ are optionally joined to form a ring system selected from substituted or unsubstituted cycloalkyl, substituted or unsubstituted heterocycloalkyl, substituted or unsubstituted aryl and substituted or unsubstituted heteroaryl. R¹⁷ and R¹⁸ are independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl; and R¹⁷ and R¹⁸, together with the atoms to which they are attached, are optionally joined to form a 5-, 6- or 7-membered ring. When A is oxygen, R⁹ is not present; and when G is oxygen, R⁷ is not present. A^{b1} and A^{b2} are attached to L¹ and L² through two members selected from R⁶, R⁷, R⁸, R⁹ and R¹⁰. A^{p1} and A^{p2} are attached to L² through a single member selected from R⁶, R⁷, R⁸, R⁹ and R¹⁰

In some embodiments, when A^{b1} has a structure according to formula (B), A^{b1} is attached to L¹ and L² through R⁶ and R¹⁰; when A^{b1} has a structure according to formula (C) or (D), A^{b1} is attached to L¹ and L² through R⁶ and R⁹; when A^{b2} has a structure according to formula (B), A^{b2} is attached to L¹ and L² through R⁶ and R¹⁰; when A^{b2} has a structure according to formula (C) or (D), A^{b2} is attached to L¹ and L² through R⁶ and R⁹; when A^{p1} has a structure according to formula (B), A^{p1} is attached to L² through R⁶ or R¹⁰; when A^{p1} has a structure according to formula (C) or (D), A^{p1} is attached to L² through R⁶ or R⁹; when A^{p2} has a structure according to formula (B), A^{p2} is attached to L² through R⁶ or R¹⁰; and when A^{p2} has a structure according to formula (C) or (D), A^{p2} is attached to L² through R⁶ or R⁹.

In various embodiments, one or more of R⁶, R⁷, R⁸, R⁹, R¹⁰ and/or R¹¹ is a linker to a reactive functional group or a linker to a targeting moiety as these moieties are described herein.

In various embodiments, A^{b1} and A^{b2} are independently selected from:

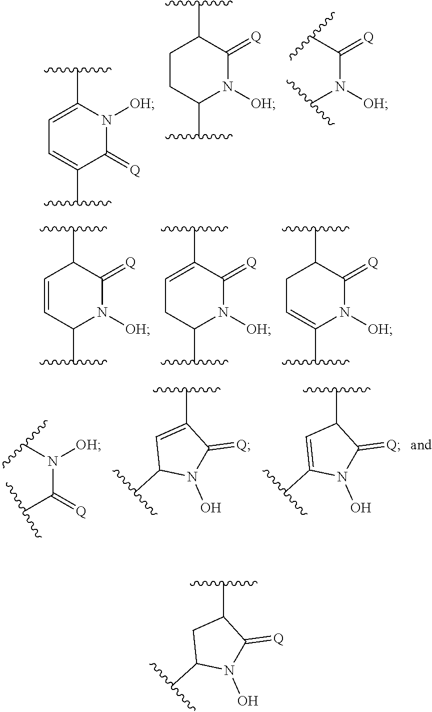

One or more of the positions occupied by H in formulae above are optionally substituted with an “aryl group substituent” or an “alkyl group substituent”, respective to whether the formula is an aryl or alkyl moiety, as these terms are defined herein. Exemplary substituents include substituted or unsubstituted alkyl, a halogen, and substituted and unsubstituted heteroalkyl, in both their linear and cyclic forms. Further exemplary substituents include a linker to a reactive functional group and/or a linker to a targeting moiety.

In some embodiments, A^{b1} and A^{b2} are each independently selected from:

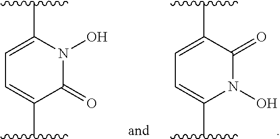 [SMILES: *c1ccc(*)n(O)c1=O ; *c1ccc(*)n(O)c1=O]

These exemplary structures provide an example of “down” and “up” orientations of examplary chelating agent/metal ion chelate of this disclosure. In an exemplary embodiment, the “down” orientation is preferred and the compounds of the invention include at least one bridging chelating moiety in the “down” orientation.

In some embodiments, A^{p1} and A^{p2} are independently selected from C₂-C₆ alkyl substituted with a member selected from:
(i) substituted or unsubstituted, saturated or unsaturated cycloalkyl;
(ii) substituted or unsubstituted, saturated or unsaturated heterocycloalkyl, each comprising an oxo and a NOH moiety;
(iii) (CH₂)ₓC(O)NR^{1a}NR^{2a}R^{3a}, (CH₂)ₓN(OH)C(O)Ria and (CH₂)ₓC(O)N(OH)R^{1a},
wherein R^{1a}, R^{2a}, and R^{3a} are independently selected from H and C₁-C₆ substituted or unsubstituted alkyl; and
and x is selected from the integers from 1 to 6.

In various embodiments, A^{p1} and A^{p2} are independently selected from:

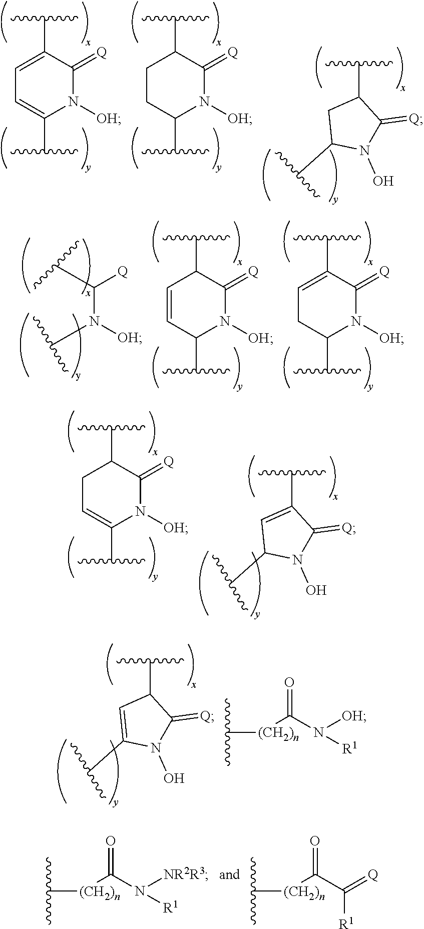

in which x and y are independently 0 or 1 with the proviso that x and y are not both 0 or both 1; Q is selected from O, S, and SH; and n is an integer from 1 to 6. This representation is intended to illustrate the two different conformations the chelating agents can assume when attached to another component of the ligand. In the figures above, the moiety terminated by the wavy line indicates a point of attachment between the chelating moiety another component of the ligand, e.g., the moiety is a bond, a methylene, ethylene, propylene, butylene or higher order divalent moiety attaching the chelating moiety to another component of the ligand. The divalent moiety can be substituted or unsubstituted.

One or more of the positions occupied by H in formulae above are optionally substituted with an “aryl group substituent” or an “alkyl group substituent”, respective to whether the formula represents an aryl or alkyl moiety, as these terms are defined herein. Exemplary substituents include substituted or unsubstituted alkyl, and substituted and unsubstituted heteroalkyl, in both their linear and cyclic forms.

Exemplary compounds of the invention include compounds of the structure according to Formula I.

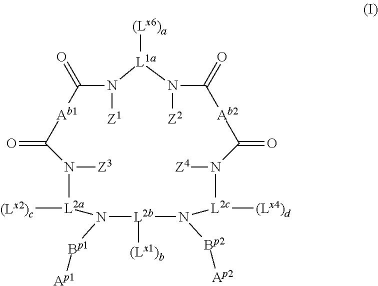

wherein L^{x1}, L^{x2}, L^{x3}, L^{x4}, L^{x6}, Z¹, Z², Z³ and Z⁴ are each independently selected from H, substituted or unsubstituted alkyl and substituted or unsubstituted heteroalkyl, or a linker to a reactive functional group or a targeting moiety. An exemplary linker is selected from a C₁-C₂₀ substituted or unsubstituted alkyl moiety, and a 1-20 atom length substituted or unsubstituted heteroalkyl moiety; L^{1a}, L^{2a}, L^{2b}, and L^{2c} are independently selected substituted or unsubstituted C₂-C₆ alkyl; each index a, b, c, or d is independently selected from 0 and 1; A^{b1} and A^{b2} are independently selected from: (i) substituted or unsubstituted, saturated or unsaturated alkyl selected from straight-chain, branched chain, and cycloalkyl; (ii) substituted or unsubstituted, saturated or unsaturated heterocycloalkyl; and (iii) substituted or unsubstituted heteroaryl, each of (i), (ii) and (iii) comprising an oxo and a NOH moiety; B^{p1} and B^{p2} are branching groups independently selected from: a bond, C₁-C₃ alkyl comprising an oxo group, and —C(O)—; A^{p1} and A^{p2} are independently selected from: (a) C₁-C₆ alkyl substituted with a member selected from: (i) substituted and unsubstituted, saturated and unsaturated cycloalkyl; (ii) substituted and unsubstituted, saturated and unsaturated heterocycloalkyl, each comprising an oxo and a NOH moiety; and (b) (CH₂)ₓC(O)NR¹NR²R³, (CH₂)ₓN(OH)C(O)R¹ and (CH₂)ₓC(O)N(OH)R¹, wherein R¹, R², and R³ are independently selected from H and C₁-C₆ substituted and unsubstituted alkyl; and x is integer selected from 1, 2 and 3. Z¹, Z², Z³ and Z⁴ are independently selected from H, substituted or unsubstituted alkyl and substituted or unsubstituted heteroalkyl.

In an exemplary embodiment, one or more of Z¹, Z², Z³ and Z⁴ is an independently selected linker as that term is defined herein. In various embodiments, one or more of Z¹, Z², Z³ and Z⁴ is an independently selected linker to a reactive functional group or to a targeting moiety. In various embodiments, the linker is substituted or unsubstituted alkyl or substituted or unsubstituted heteroalkyl. An exemplary reactive functional group to which the linker is attached is an amine, an alcohol, a carboxylic acid, or a reactive derivative thereof (e.g., maleimide, isocyanate, isothiocyanate), ester (e.g., active ester), or other reactive functional groups set forth herein. In exemplary embodiments, the reactive functional group is a halide (e.g., acyl halide).

In an exemplary embodiment, each a, b, c, and d is 0, or each L^{x1}, L^{x2}, L^{x4} and L^{x6} is H, Z¹-Z⁴ are each H, and the ligand does not have a linker to a reactive functional group or a linker to a targeting moiety. In some embodiments, one or more of the index a, b, c, or d is 1, at least one L^{x6} is other than H, and the ligand includes one or more linker to a reactive functional group or a linker to a targeting moiety. In some embodiments, each a, b, c, and d is 0, or each L^{x1}, L^{x2}, L^{x4} and L^{x6} is H, and one or more of Z¹-Z⁴ is a linker to a reactive functional group or a linker to a targeting moiety.

In some embodiments, the chelating agent/metal ion chelate of the invention is of a structure according to Formula II:

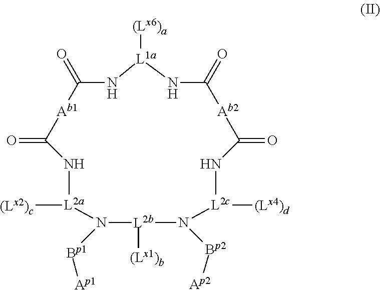

in which the various moieties are as described above.

In some embodiments, a member selected from A^{b1} and A^{b2} and a combination thereof is not a phenyl moiety substituted with one or more OH. In an exemplary embodiment, a member selected from A^{b1} and A^{b2} and a combination thereof is not:

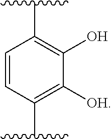 [SMILES: *c1ccc(*)c(O)c1O]

In some embodiments, A^{b1} and A^{b2} are each independently selected from:

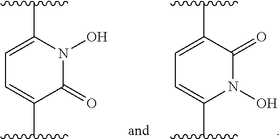 [SMILES: *c1ccc(*)n(O)c1=O ; *c1ccc(*)n(O)c1=O]

The formulae shown immediately above are exemplars of two configurations of an exemplary bridging chelating moiety, “down” and “up”. In an exemplary embodiment, the “down” orientation is preferred and the compounds of the invention include at least one bridging chelating moiety in the “down” orientation.

In some embodiments, A^{p1} and A^{p2} are each independently selected from:

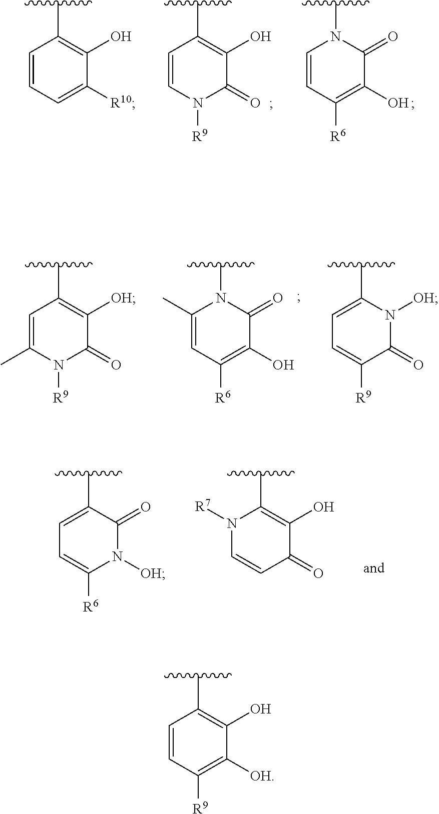

In some embodiments, A^{p1} and A^{p2} are each independently selected from:

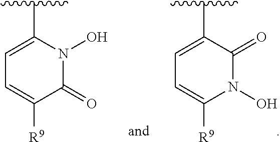

In some embodiments, A^{p1} and A^{p2} each are:

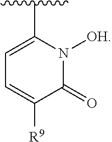

In some embodiments, one or both of A^{p1} and A^{p2} comprise a modifying moiety. Modifying moieties are as defined herein. In some embodiments, R⁹ of A^{p1}, A^{p2}, or A^{p1} and A^{p2} comprises a modifying moiety. In some embodiments, R⁹ of A^{p1}, A^{p2}, or A^{p1} and A^{p2} is —C(O)NR¹⁷R¹⁸, wherein R¹⁷ is H and R¹⁸ is a modifying moiety. In some embodiments, R⁶ of A^{p1}, A^{p2}, or A^{p1} and A^{p2} comprises a modifying moiety. In some embodiments, R⁶ of API, A^{p2}, or A^{p1} and A^{p2} is —C(O)NR¹⁷R¹⁸, wherein R¹⁷ is H and R¹⁸ is a modifying moiety.

### Linker to Functional/Targeting Moiety

A “linker”, “linking member”, or “linking moiety”, e.g., L^{x1}, L^{x2}, L^{x4}, L^{x6}, Z¹, Z², Z³ and Z⁴ as used herein is a moiety that joins or potentially joins, covalently or noncovalently, a first moiety to a second moiety within the chelating agent/metal ion chelate structure. In an exemplary embodiment, a linker attaches or could potentially attach a chelating agent/metal ion chelate described herein to another molecule, such as a targeting moiety. In some embodiments, a linker attaches or could potentially attach a ligand described herein to a solid support. A linker comprising a reactive functional group that can be further reacted with a reactive functional group on a structure of interest in order to attach the structure of interest to the linker is referred to as a “functionalized linker”, or a “linker to a reactive functional group”. In exemplary embodiments, a linker is a functionalized linker. In exemplary embodiments, a ligand comprises one or more functionalized linkers. In some embodiments, a linker comprises a targeting moiety. In some embodiments, a linker to a targeting moiety comprises a bond to the targeting moiety.

In some embodiments, the linker is a linker to a reactive functional group, or a linker to a targeting moiety. In some embodiments, the reactive functional group is a protected functional group.

A linker can be any useful structure incorporating a reactive functional group or a targeting moiety, such as an antibody, into a chelating agent/metal ion chelate of the invention. Examples of a linker include 0-order linkers (i.e., a bond), substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. Further exemplary linkers include substituted or unsubstituted (C₁, C₂, C₃, C₄, C₅, C₆, C₇, C₈, C₉ or C₁₀) alkyl, substituted or unsubstituted heteroalkyl, —C(O)NR′—, —C(O)O—, —C(O)S—, and —C(O)CR′R″, wherein R′ and R″ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl. In some embodiments, a linker includes at least one heteroatom. Exemplary linkers also include —C(O)NH—, —C(O), —NH—, —S—, —O—, and the like. In an exemplary embodiment, a linker is a heteroalkyl substituted with a reactive functional group. A linker to a reactive functional group or to a targeting moiety can be placed at any location on the chelating agent/metal ion chelate framework.

### Reactive Functional Groups

In one embodiment, a linker comprises a reactive functional group (or a “reactive functional moiety”, used synonymously), which can be further reacted to covalently attach the linker to a targeting or other moiety. Reactive functional groups and classes of reactions useful in practicing the present invention are generally those that are well known in the art of bioconjugate chemistry. Currently favored classes of reactions available with reactive functional groups of the invention are those which proceed under relatively mild conditions. These include, but are not limited to nucleophilic substitutions (e.g., reactions of amines and alcohols with acyl halides and activated esters), electrophilic substitutions (e.g., enamine reactions) and additions to carbon-carbon and carbon-heteroatom multiple bonds (e.g., Michael reactions and Diels-Alder reactions). These and other useful reactions are discussed, for example, in March, Advanced Organic Chemistry (3rd Ed., John Wiley & Sons, New York, 1985); Hermanson, Bioconjugate Techniques (Academic Press, San Diego, 1996); and Feeney et al., Modification of Proteins, Advances in Chemistry Series, Vol. 198 (American Chemical Society, Washington, D.C., 1982).

The reactive functional groups set forth in this disclosure can be used in any combination that provides a desired characteristic of a chelating agent/metal ion chelate of this disclosure. Where reactive functional groups are represented at a particular locus, the identity of the reactive functional group is intended to be illustrative, not thereby limiting the disclosure to that particular reactive functional group at that particular locus.

In some embodiments, a reactive functional group refers to a group selected from olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids thiohydroxamic acids, allenes, ortho esters, sulfites, enamines, ynamines, ureas, pseudoureas, semicarbazides, carbodiimides, carbamates, imines, azides, azo compounds, azoxy compounds, and nitroso compounds. Reactive functional groups also include those used to prepare bioconjugates, e.g., N-hydroxysuccinimide esters, maleimides and the like. Methods to prepare each of these functional groups are well known in the art and their application or modification for a particular purpose is within the ability of one of skill in the art (see, for example, Sandler and Karo, eds., Organic Functional Group Preparations, (Academic Press, San Diego, 1989)).

A reactive functional group can be chosen according to a selected reaction partner. As an example, an activated ester, such as an NHS ester will be useful to label a protein via lysine residues. Sulfhydryl reactive groups, such as maleimides can be used to label proteins via amino acid residues carrying an SH-group (e.g., cystein). Antibodies may be labeled by first oxidizing their carbohydrate moieties (e.g., with periodate) and reacting resulting aldehyde groups with a hydrazine containing chelating agent/metal ion chelate.

The reactive functional groups can be chosen such that they do not participate in, or interfere with, the reactions necessary to assemble the reactive chelating agent/metal ion chelate. Alternatively, a reactive functional group can be protected from participating in the reaction by means of a protecting group. Those of skill in the art understand how to protect a particular functional group so that it does not interfere with a chosen set of reaction conditions. For examples of useful protecting groups, see, for example, Greene et al., PROTECTIVE GROUPS IN ORGANIC SYNTHESIS, John Wiley & Sons, New York, 1991.

### Amines and Amino-Reactive Groups

In one embodiment, a reactive functional group is selected from an amine, (such as a primary or secondary amine), hydrazine, hydrazide and sulfonylhydrazide. Amines can, for example, be acylated, alkylated or oxidized. Useful non-limiting examples of amino-reactive groups include N-hydroxysuccinimide (NHS) esters, sulfur-NHS esters, imidoesters, isocyanates, isothiocyanates, acylhalides, arylazides, p-nitrophenyl esters, aldehydes, sulfonyl chlorides, thiazolides and carboxyl groups.

NHS esters and sulfo-NHS esters react preferentially with primary (including aromatic) amino groups of a reaction partner. The imidazole groups of histidines are known to compete with primary amines for reaction, but the reaction products are unstable and readily hydrolyzed. The reaction involves the nucleophilic attack of an amine on the acid carboxyl of an NHS ester to form an amide, releasing the N-hydroxysuccinimide.

Imidoesters are the most specific acylating reagents for reaction with amine groups of a molecule such as a protein. At a pH between 7 and 10, imidoesters react only with primary amines. Primary amines attack imidates nucleophilically to produce an intermediate that breaks down to amidine at high pH or to a new imidate at low pH. The new imidate can react with another primary amine, thus crosslinking two amino groups, a case of a putatively monofunctional imidate reacting bifunctionally. The principal product of reaction with primary amines is an amidine that is a stronger base than the original amine. The positive charge of the original amino group is therefore retained. As a result, imidoesters do not affect the overall charge of the conjugate.

Isocyanates (and isothiocyanates) react with the primary amines of the conjugate components to form stable bonds. Their reactions with sulfhydryl, imidazole, and tyrosyl groups give relatively unstable products.

Acylazides are also used as amino-specific reagents in which nucleophilic amines of the reaction partner attack acidic carboxyl groups under slightly alkaline conditions, e.g. pH 8.5.

Arylhalides such as 1,5-difluoro-2,4-dinitrobenzene react preferentially with the amino groups and tyrosine phenolic groups of the conjugate components, but also with its sulfhydryl and imidazole groups.

p-Nitrophenyl esters of carboxylic acids are also useful amino-reactive groups. Although the reagent specificity is not very high, α- and ε-amino groups appear to react most rapidly.

Aldehydes react with primary amines of the conjugate components (e.g., ε-amino group of lysine residues). Although unstable, Schiff bases are formed upon reaction of the protein amino groups with the aldehyde. Schiff bases, however, are stable, when conjugated to another double bond. The resonant interaction of both double bonds prevents hydrolysis of the Schiff linkage. Furthermore, amines at high local concentrations can attack the ethylenic double bond to form a stable Michael addition product. Alternatively, a stable bond may be formed by reductive amination.

Aromatic sulfonyl chlorides react with a variety of sites of the conjugate components, but reaction with the amino groups is the most important, resulting in a stable sulfonamide linkage.

Free carboxyl groups react with carbodiimides, soluble in both water and organic solvents, forming pseudoureas that can then couple to available amines yielding an amide linkage. Yamada et al., Biochemistry, 1981, 20: 4836-4842, e.g., teach how to modify a protein with carbodiimides.

### Sulfhydryl and Sulfhydryl-Reactive Groups

In another embodiment, a reactive functional group is selected from a sulfhydryl group (which can be converted to disulfides) and sulfhydryl-reactive group. Useful non-limiting examples of sulfhydryl-reactive groups include maleimides, alkyl halides, acyl halides (including bromoacetamide or chloroacetamide), pyridyl disulfides, and thiophthalimides.

Maleimides react preferentially with the sulfhydryl group of the conjugate components to form stable thioether bonds. They also react at a much slower rate with primary amino groups and the imidazole groups of histidines. However, at pH 7 the maleimide group can be considered a sulfhydryl-specific group, since at this pH the reaction rate of simple thiols is 1000-fold greater than that of the corresponding amine.

Alkyl halides react with sulfhydryl groups, sulfides, imidazoles, and amino groups. At neutral to slightly alkaline pH, however, alkyl halides react primarily with sulfhydryl groups to form stable thioether bonds. At higher pH, reaction with amino groups is favored.

Pyridyl disulfides react with free sulfhydryl groups via disulfide exchange to give mixed disulfides. As a result, pyridyl disulfides are relatively specific sulfhydryl-reactive groups.

Thiophthalimides react with free sulfhydryl groups to also form disulfides.

### Other Reactive Functional Groups

Other exemplary reactive functional groups include:
(i) carboxyl groups and various derivatives thereof including, but not limited to, N-hydroxybenztriazole esters, acid halides, acyl imidazoles, thioesters, p-nitrophenyl esters, alkyl, alkenyl, alkynyl and aromatic esters;
(ii) hydroxyl groups, which can be converted to esters, ethers, aldehydes, etc.;
(iii) haloalkyl groups, wherein the halide can be displaced with a nucleophilic group such as, for example, an amine, a carboxylate anion, thiol anion, carbanion, or an alkoxide ion, thereby resulting in the covalent attachment of a new group at the site of the halogen atom;
(iv) dienophile groups, which are capable of participating in Diels-Alder reactions such as, for example, maleimido groups;
(v) aldehyde or ketone groups, such that subsequent derivatization is possible via formation of carbonyl derivatives such as, for example, imines, hydrazones, semicarbazones or oximes, or via such mechanisms as Grignard addition or alkyllithium addition;
(vi) alkenes, which can undergo, for example, cycloadditions, acylation, Michael addition, etc;
(vii) epoxides, which can react with, for example, amines and hydroxyl groups; (ix) phosphoramidites and other standard functional groups useful in nucleic acid synthesis and
(x) any other functional group useful to form a covalent bond between the functionalized ligand chelating agent/metal ion chelate and a molecular entity or a surface.
   Functional Groups with Non-Specific Reactivities

In addition to the use of site-specific reactive moieties, the present invention contemplates the use of non-specific reactive groups to link a ligand chelating agent/metal ion chelate to a targeting moiety. Non-specific groups include photoactivatable groups, for example.

Photoactivatable groups are ideally inert in the dark and are converted to reactive species in the presence of light. In one embodiment, photoactivatable groups are selected from precursors of nitrenes generated upon heating or photolysis of azides. Electron-deficient nitrenes are extremely reactive and can react with a variety of chemical bonds including N—H, O—H, C—H, and C=C. Although three types of azides (aryl, alkyl, and acyl derivatives) may be employed, arylazides are presently preferred. The reactivity of arylazides upon photolysis is better with N—H and O—H than C—H bonds. Electron-deficient arylnitrenes rapidly ring-expand to form dehydroazepines, which tend to react with nucleophiles, rather than form C—H insertion products. The reactivity of arylazides can be increased by the presence of electron-withdrawing substituents such as nitro or hydroxyl groups in the ring. Such substituents push the absorption maximum of arylazides to longer wavelength. Unsubstituted arylazides have an absorption maximum in the range of 260-280 nm, while hydroxy and nitroarylazides absorb significant light beyond 305 nm. Therefore, hydroxy and nitroarylazides are most preferable since they allow to employ less harmful photolysis conditions for the affinity component than unsubstituted arylazides.

In another preferred embodiment, photoactivatable groups are selected from fluorinated arylazides. The photolysis products of fluorinated arylazides are arylnitrenes, all of which undergo the characteristic reactions of this group, including C—H bond insertion, with high efficiency (Keana et al., J. Org. Chem. 55: 3640-3647, 1990).

In another embodiment, photoactivatable groups are selected from benzophenone residues. Benzophenone reagents generally give higher crosslinking yields than arylazide reagents.

In another embodiment, photoactivatable groups are selected from diazo compounds, which form an electron-deficient carbene upon photolysis. These carbenes undergo a variety of reactions including insertion into C—H bonds, addition to double bonds (including aromatic systems), hydrogen attraction and coordination to nucleophilic centers to give carbon ions.

In still another embodiment, photoactivatable groups are selected from diazopyruvates. For example, the p-nitrophenyl ester of p-nitrophenyl diazopyruvate reacts with aliphatic amines to give diazopyruvic acid amides that undergo ultraviolet photolysis to form aldehydes. The photolyzed diazopyruvate-modified affinity component will react like formaldehyde or glutaraldehyde forming intraprotein crosslinks.

In some embodiments, a linker joins a chelating agent/metal ion chelate to a reactive functional group. In exemplary embodiments, a linker joins a chelating agent/metal ion chelate to a targeting moiety. That is, in exemplary embodiments, a linker comprises a targeting moiety. In some embodiments, a chelating agent/metal ion chelate comprises a linker to a targeting moiety. Any linker described herein may be a linker comprising a reactive functional group that could react with a reactive functional group on a targeting moiety to join the linker to the targeting moiety. Any linker described herein may be a linker comprising a bond to a targeting moiety. The term “targeting moiety” refers to a moiety serves to target or direct the molecule to which it is attached (e.g., a chelating agent/metal ion chelate (such as a chelate of a radionuclide)) to a particular location or molecule. Thus, for example, a targeting moiety may be used to target a chelating agent/metal ion chelate to a specific target protein or enzyme, or to a particular cellular location, to a particular cell type or to a diseased tissue. As will be appreciated by those in the art, the localization of proteins within a cell is a simple method for increasing effective concentration. For example, shuttling an imaging agent and/or therapeutic based on a chelating agent/metal chelate of this disclosure into the nucleus confines them to a smaller space thereby increasing concentration. Finally, the physiological target may simply be localized to a specific compartment, and the chelating agent/metal ion chelate is desirably localized appropriately.

The targeting moiety can be a small molecule (e.g., MW<500D), which includes both non-peptides and peptides. Examples of a targeting moiety also include peptides, polypeptides (including proteins, and in particular antibodies, which includes antibody fragments), nucleic acids, oligonucleotides, carbohydrates, lipids, hormones (including proteinaceous and steroid hormones (for instance, estradiol)), growth factors, lectins, receptors, receptor ligands, cofactors and the like. Targets of a targeting moiety can include a complementary nucleic acid, a receptor, an antibody, an antigen or a lectin, for example.

In exemplary embodiments, a targeting moiety can bind to a target with high binding affinity. In other words, a targeting moiety with high binding affinity to a target has a high specificity for or specifically binds to the target. In some embodiments, a high binding affinity is given by a dissociation constant K_{d} of about 10⁻⁷ M or less. In exemplary embodiments, a high binding affinity is given by a dissociation constant K_{d} of about 10⁻⁸ M or less, about 10⁻⁹ M or less, about 10⁻¹⁰ M or less, about 10⁻¹¹ M or less, about 10⁻¹² M or less, about 10⁻¹³ M or less, about 10⁻¹⁴ M or less or about 10⁻¹⁵ M or less. A compound may have a high binding affinity for a target if the compound comprises a portion, such as a targeting moiety, that has a high binding affinity for the target.

In exemplary embodiments, a targeting moiety is an antibody. An “antibody” refers to a protein comprising one or more polypeptides that is or can be substantially encoded by all or part of the recognized immunoglobulin genes. The recognized immunoglobulin genes, for example in humans, include the kappa (κ), lambda (λ) and heavy chain genetic loci, which together compose the myriad variable region genes, and the constant region genes mu (μ), delta (δ), gamma (γ), epsilon (ε) and alpha (α), which encode the IgM, IgD, IgG, IgE, and IgA isotypes respectively. Antibody herein is meant to include full length antibodies and antibody fragments, and may refer to a natural antibody from any organism, an engineered antibody or an antibody generated recombinantly for experimental, therapeutic or other purposes as further defined below. Antibody fragments include Fab, Fab′, F(ab′)₂, Fv, scFv or other antigen-binding subsequences of antibodies and can include those produced by the modification of whole antibodies or those synthesized de novo using recombinant DNA technologies. The term “antibody” refers to both monoclonal and polyclonal antibodies. Antibodies can be antagonists, agonists, neutralizing, inhibitory or stimulatory.

While a targeting moiety may be appended to a chelating agent/metal ion chelate/metal ion chelate in order to localize the compound to a specific region in an animal, certain chelating agents/metal ion chelates have a natural affinity for certain cells, tissue, organs or some other part of the subject to whom they are administered. For example, a ligand disclosed herein might have a natural or intrinsic affinity for bone. Thus, in some embodiments, a chelating agent/metal ion chelate does not comprise a targeting moiety or a linker to a targeting moiety, yet by its structure it inherently localizes at a desired target. In some embodiments, a chelating agent/metal ion chelate lacking a targeting moiety is used in a method not requiring specific targeting.

In some embodiments, a chelating agent/metal ion chelate comprises a linker to a solid support. That is, any linker described herein may be a linker comprising a reactive functional group that could react with a reactive functional group on a solid support to join the linker to the solid support. Any linker described herein may be a linker comprising a bond to a solid support. A “solid support” is any material that can be modified to contain discrete individual sites suitable for the attachment or association of a chelating agent/metal ion chelate. Suitable substrates include biodegradable beads, non-biodegradable beads, silica beads, magnetic beads, latex beads, glass beads, quartz beads, metal beads, gold beads, mica beads, plastic beads, ceramic beads, or combinations thereof. Of particular use are biocompatible polymers, including biodegradable polymers that are slowly removed from the system by enzymatic degradation. Example biodegradable materials include starch, cross-linked starch, poly(ethylene glycol), polyvinylpyrrolidine, polylactides (PLA), polyglycolides (PGA), poly(lactide-co-glycolides) (PLGA), polyanhydrides, polyorthoesters, poly(DTH iminocarbonate), poly(bisphenol A iminocarbonate), polycyanoacrylate, polyphosphazene, mixtures thereof and combinations thereof. Other suitable substances for forming the particles exist and can be used. In some embodiments, a solid support is a bead comprising a cross-linked starch, for example, cross-linked potato starch. Beads made from starch are completely biodegradable in the body, typically by serum amylase, a naturally occurring enzyme found in the body. In these embodiments, the chelating agent/metal ion chelate optionally further comprises a targeting moiety or a linker to a targeting moeity. In cases where a chelating agent/metal ion chelate that is attached to a solid support does not comprise a targeting moiety, the chelating agent/metal ion chelate can be localized directly by the practitioner, for example, by direct surgical implantation.

In some embodiments, a linker is bound to the moiety F^{x}, e.g., having the structure -L^{x6}-F^{x}, wherein the linker, e.g., L^{x6}, is selected from a bond, acyl, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl; and F^{x} is selected from a reactive functional group, a protected functional group, or a targeting moiety.

In some embodiments, one or more L^{x} linker, e.g., L^{x2}, L^{x4}, or L^{x6}, is selected from substituted or unsubstituted alkyl and substituted or unsubstituted heteroalkyl. In some embodiments, L^{x6} is heteroalkyl. In some embodiments, L^{x6} is (C₁, C₂, C₃, C₄, C₅, C₆, C₇, C₈, C₉, C₁₀, C₁₁, C₁₂, C₁₃, C₁₄, C₁₅, C₁₆, C₁₇, Cis, C₁₉ or C₂₀) alkyl in which 1, 2 or 3 atoms are replaced with a heteroatom, such as nitrogen or oxygen. In some embodiments, L^{x6} comprises a modifying moiety.

In some embodiments, F^{x} is selected from —NH₂, —C(O)OH, alkyl ester (e.g., methyl ester), N-hydroxysuccinimide (NHS) ester, sulfo-NHS ester, isothiocyanate, and maleimide. In some embodiments, F^{x} is selected from —NH₂ and —C(O)OH.

In some embodiments, one or more of the L^{x} linkers, e.g., L^{x2}-F^{x}, L^{x4}-F^{x} or L^{x6}-F^{x} is selected from:

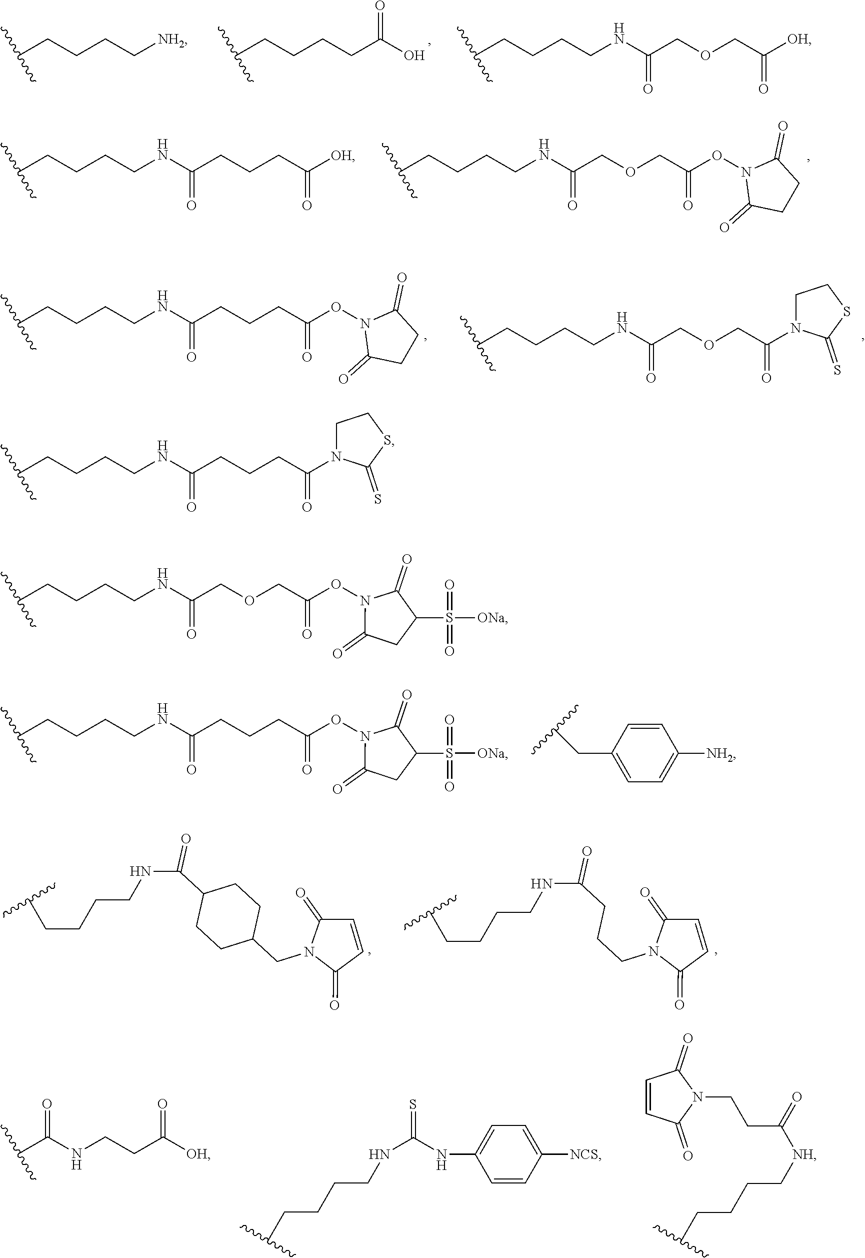

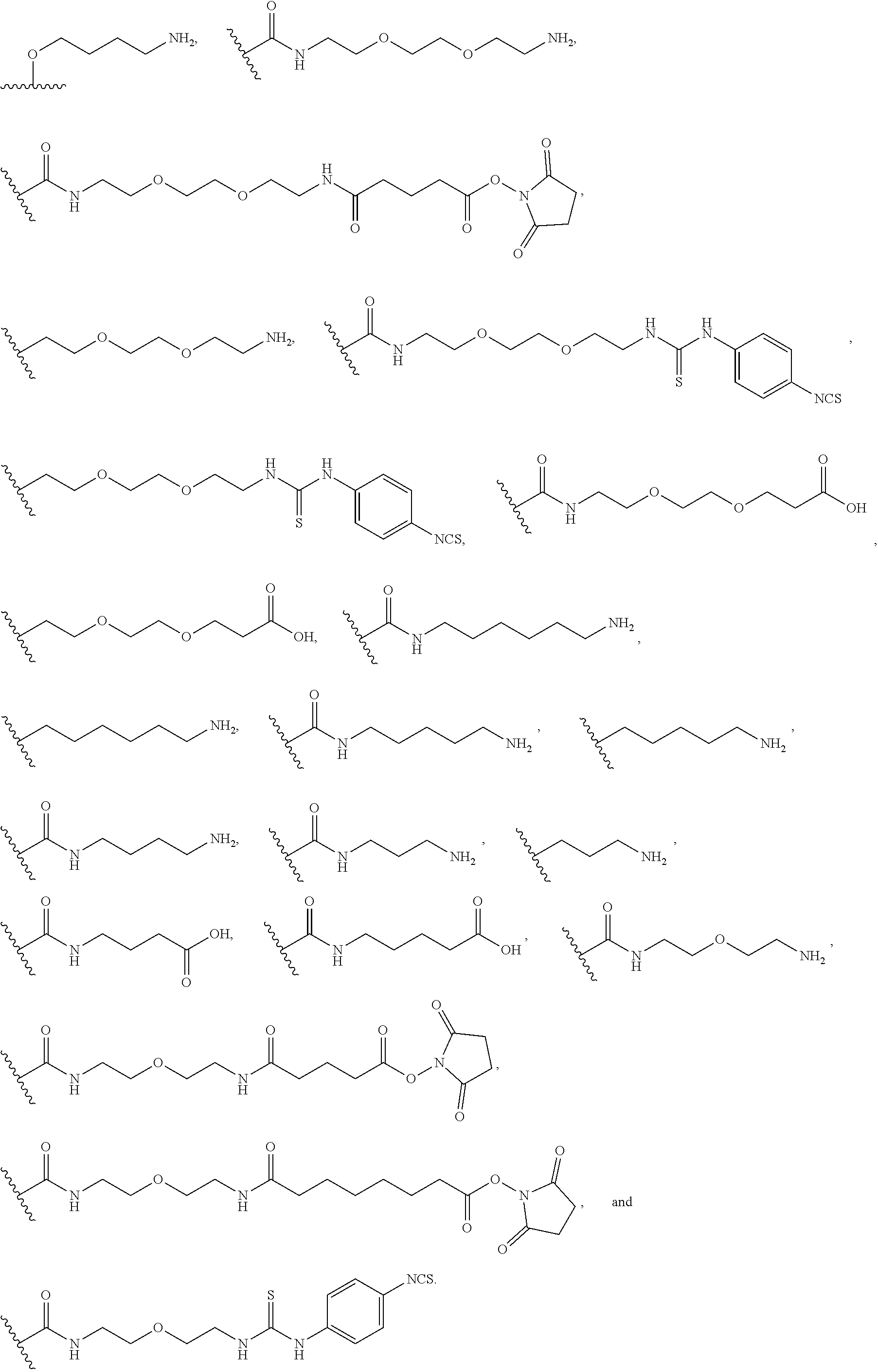

In an exemplary embodiment, any implied hydrogens in structure in this disclosure can be selected from substituted or unsubstituted alkyl, and substituted or unsubstituted heteroalkyl of 1, 2, 3, 4, 5, 6, 7, 8, 9 members selected from C or a heteroatom.

In some embodiments, a linker has the structure:

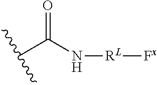

wherein R^{L} is selected from substituted or unsubstituted alkyl, and substituted or unsubstituted heteroalkyl; and F^{x} is as defined herein. In some embodiments, R^{L} is a substituted or unsubstituted alkoxyalkyl. In some embodiments, R^{L} is a substituted or unsubstituted monoether. In some embodiments, R^{L} is a substituted or unsubstituted polyether. In some embodiments, the polyether has from 2 to 10 (i.e., 2, 3, 4, 5, 6, 7, 8, 9, or 10) ether groups. In some embodiments, R^{L} comprises a modifying moiety.

In some embodiments, a linker has the structure:

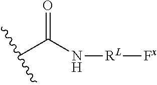

wherein R^{L} is selected from substituted or unsubstituted alkyl or substituted or unsubstituted heteroaryl, e.g., C₁, C₂, C₃, C₄, C₅ or C₆ alkyl, or a 2-, 3-, 4-, 5-, 6-membered substituted or unsubstituted heteroalkyl, including, for example:

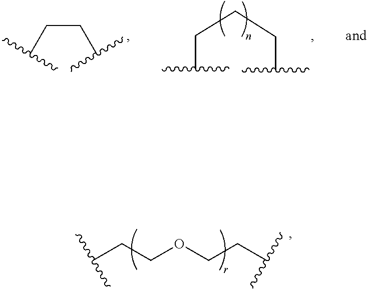

wherein n is an integer selected from 0, 1, 2, 3, 4, 5, and 6; r is an integer selected from 1, 2, 3, 4, 5, and 6; and F^{x} is a reactive functional group (such as NH₂) or a protected functional group.

In some embodiments, a linker-F^{x} moiety has a structure selected from:

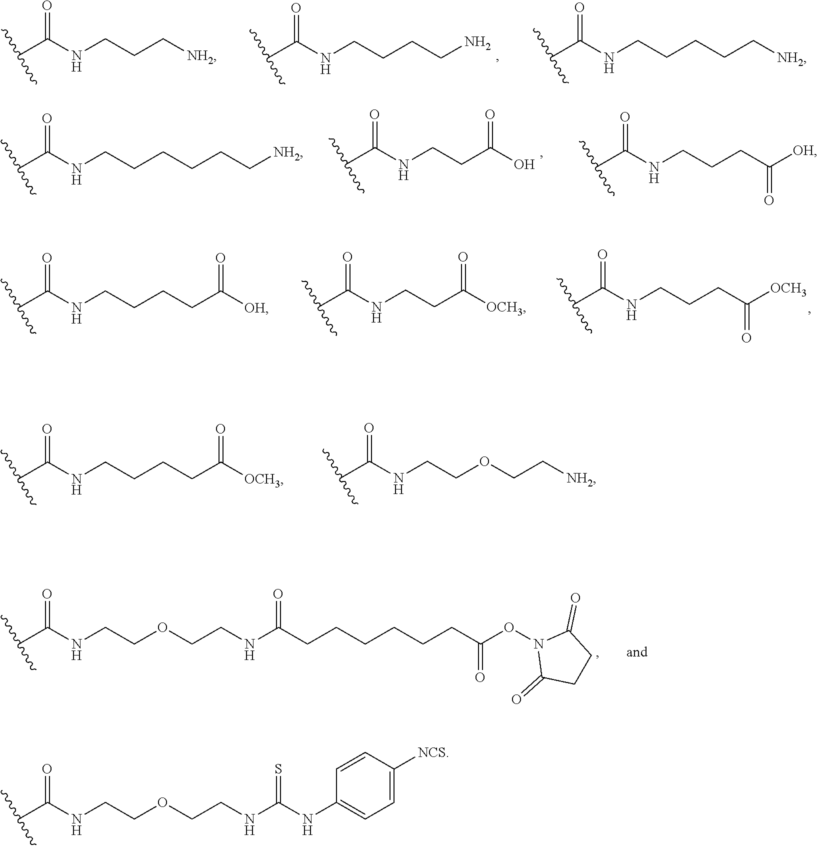

In an exemplary embodiment, a linker-F moiety has a structure selected from:

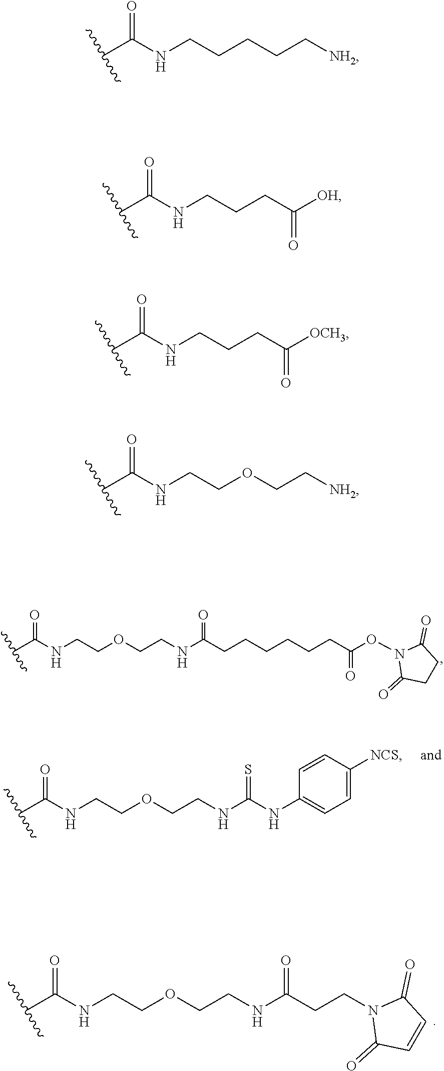

In various embodiments, a linker —F^{x} moiety has a structure selected from:

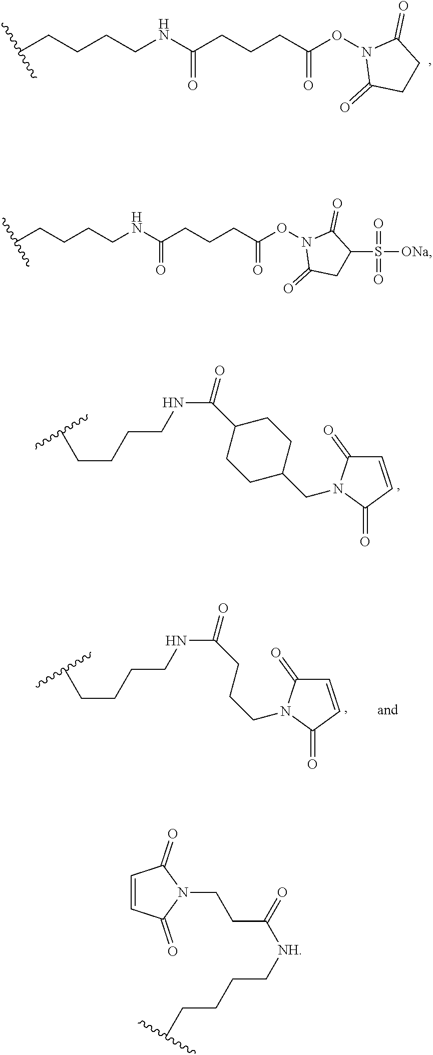

In an exemplary embodiment, a linker-F moiety has a structure selected from:

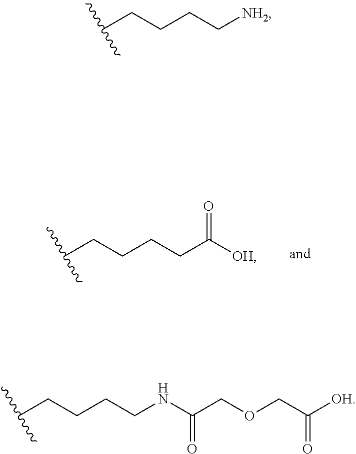 [SMILES: *CCCCN ; *CCCCC(=O)O ; *CCCCNC(=O)COCC(=O)O]

In exemplary embodiments, F^{x} is a targeting moiety.

In exemplary embodiments, a linker is a linker to a targeting moiety. In some embodiments, the targeting moiety is selected from a polypeptide, a nucleic acid, a lipid, a polysaccharide, a small molecule, a cofactor and a hormone. In exemplary embodiments, the targeting moiety is an antibody or antibody fragment.

In a linker with multiple reactive functional groups, a particular functional group can be chosen such that it does not participate in, or interfere with, the reaction controlling the attachment of the functionalized spacer component to another chelating agent/metal ion chelate component. Alternatively, the reactive functional group can be protected from participating in the reaction by the presence of a protecting group. Those of skill in the art understand how to protect a particular functional group from interfering with a chosen set of reaction conditions. For examples of useful protecting groups, See Greene et al., PROTECTIVE GROUPS IN ORGANIC SYNTHESIS, John Wiley & Sons, New York, 1991.

### Modifying Moiety

In some embodiments, the compound (chelating agent/metal ion chelate) comprises one or more a modifying moieties. In some embodiments, one or more of L¹, L², L^{x6}, A^{b1}, A^{b2}, A^{p1}, and A^{p2} comprise(s) a modifying moiety. In some embodiments, one or more of L^{1a}, L^{2a}, L^{2b}, L^{2c}, L^{x6}, B^{p1}, and B^{p2}, A^{b1}, A^{b2}, A^{p1}, and A^{p2} comprise(s) a modifying moiety. In some embodiments, a linker to a reactive functional group, or a linker to a targeting moiety comprises a modifying moiety. Each of the modifying moieties can be the same or different.

The modifying moiety modifies various properties of the chelating agent/metal ion chelate, such as solubility, charge, or target affinity. In some embodiments, the modifying moiety does not interact with the metal when the chelating agent/metal ion chelate is complexed to a metal. In some embodiments, the modifying moiety is a solubilizing group (e.g., a water-soluble moiety, e.g., a saccharide (e.g., sugar, sugar alcohol), poly(ether), peptides, an oligonucleotide etc.), a human hormonemoiety (recombinant, synthetic or naturally occurring), e.g., estrogen, estradiol, estriol, estrone, progesterone, testosterone, insulin, cortisol, growth hormone, adrenaline, or a thyroid hormone, a prodrug moiety (for example, with a cleavable moiety), a nucleic acid, an oligonucleotide, ssDNA, dsDNA, RNA, or a peptide. Methods to functionalize parent compounds of these species to allow their ready conjugation to a reactive functional group, such as those on examplary chelates of this disclosure, are well-know and readily available to the ordinarily skilled worker.

In exemplary embodiments, the solubilizing group improves solubility of the chelating agent/metal ion chelate and/or a complex formed between the chelating agent/metal ion chelate and a metal ion in aqueous media. Exemplary solubilizing moieties are themselves soluble in water and include moieties such as poly(ethers), oligonucleotides, peptides and saccharides (e.g., sugar, sugar alcohol). In some embodiments, the hormone is a steroid. In some embodiments, the steroid is estradiol. In some embodiments, the modifying moiety is an estradiol moiety. Modifying groups are of use to tune the solubility of the a chelating agent of a chelate of a metal ion formed with a chelate of the disclosure. In an exemplary embodiment, a peptide of a mixed hydrophilic and hydrophobic nature, by virtue of its amino acid composition, is used to tune solubility of the chelating agent/metal ion chelate.

In some embodiments, the modifying moiety is substituted or unsubstituted heteroalkyl. In some embodiments, the modifying moiety is a substituted or unsubstituted alkoxyalkyl. In some embodiments, the modifying moiety is a substituted or unsubstituted monoether. In some embodiments, the modifying moiety is a substituted or unsubstituted polyether, e.g., poly(ethylene glycol). In some embodiments, the modifying moiety comprises an estradiol moiety. In some embodiments, the modifying moiety is a polyether substituted with an estradiol moiety.

In some embodiments, the modifying moiety is selected from:

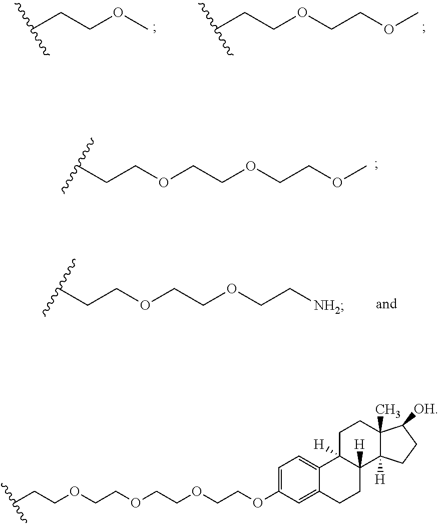

In these representations via chemical formulae, the number of repeating ethylene oxide subunits is purely illustrative. As will be appreciated the appropriate number of subunits is selectable by the person of ordinary skill in the art for a desired outcome. An exemplary moiety according to this description includes from 1 to about 100 ethylene oxide subunits. This range encompassed all individual values and subranges encompassed therewithin, e.g., 1, 3, 5, 6, 7, 8, 9, 10 . . . , from 1 to about 10, from about 3 to about 7, from about 5 to about 15, from about 25 to about 50, etc.

In some embodiments, the modifying moiety is a peptide. In some embodiments, the modifying moiety is:

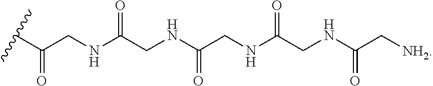 [SMILES: *C(=O)CNC(=O)CNC(=O)CNC(=O)CNC(=O)CN]

In some embodiments, the modifying moiety comprises an oligonucleotide.

In some embodiments, the modifying moiety is selected from:

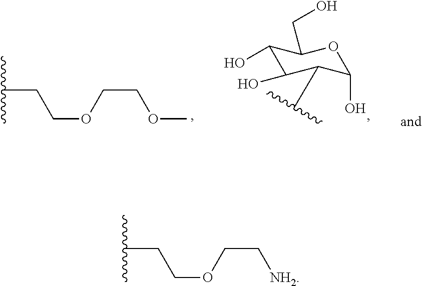

### Exemplary Chelating Agents/Metal Ion Chelates

In some embodiments, the invention provides a chelating agent/metal ion chelate having the structure:

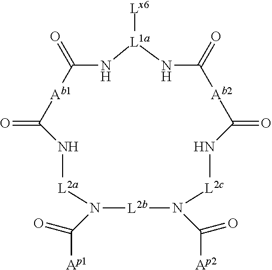

wherein L^{1a}, L^{2a}, L^{2b}, L^{2c}, L^{x6}, A^{b1}, A^{b2}, A^{p1}, and A^{p2} are as defined herein.

In some embodiments, the invention provides a chelating agent/metal ion chelate having the structure:

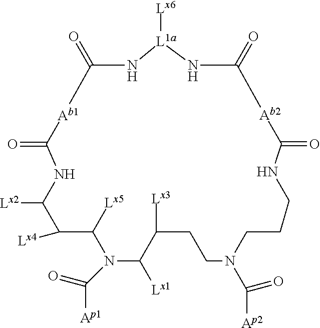

wherein L^{x1}, L^{x2}, L^{x3}, L^{x4}, L^{x5}, and L^{x6} are linkers to a reactive functional group or to a targeting moiety as defined herein. Each of these linkers can be present or absent in a chelating agent/metal ion chelate of the invention in any number and positional permutation, each of which is readily recognizable to one of skill in the art. In an exemplary embodiment, at least one of L^{x1}, L^{x2}, L^{x3}, L^{x4}, L^{x5}, and L^{x6} is present. In an exemplary embodiment, at least L^{x6} is present. In an exemplary embodiment, at least two or at least three of these linkers are present. A^{b1}, A^{b2}, A^{p1}, and A^{p2} are as defined herein.

In various embodiments, the invention provides a chelating agent/metal ion chelate having the formula:

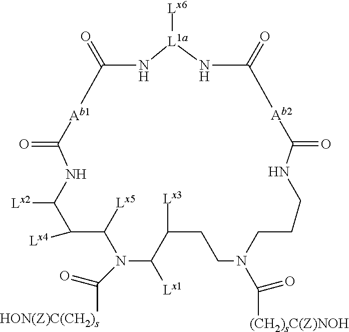

wherein L^{1a}, L^{x1}, L^{x2}, L^{x3}, L^{x4} L^{x5}, and L^{x6} are linkers to a reactive functional group or to a targeting moiety as defined herein. Each of these linkers can be present or absent in a chelating agent/metal ion chelate of the invention. In an exemplary embodiment, at least one of L^{1a}, L^{x1}, L^{x2}, L^{x3}, L^{x4}, L^{x5}, and L^{x6} is present. In an exemplary embodiment, at least L^{x6} is present. In an exemplary embodiment, at least two or at least three of these linkers are present. A^{b1}, and A^{b2} are as defined herein. Z is O or S, and each index s is independently selected from the integers 1, 2, 3, 4, 5, and 6.

In some embodiments, the invention provides a chelating agent/metal ion chelate having the structure:

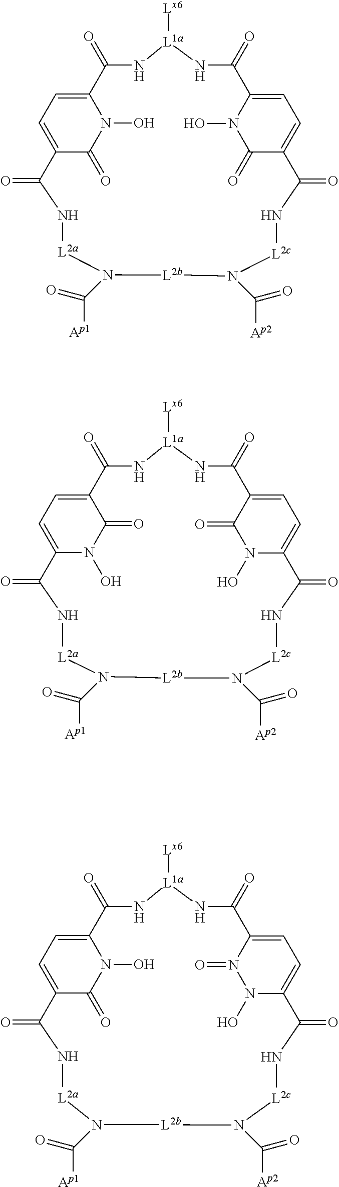

wherein L^{1a}, L^{2a}, L^{2b}, L^{2c}, L^{x6}, A^{p1}, and A^{p2} are as defined herein.

In some embodiments, the invention provides a chelating agent/metal ion chelate of the formula:

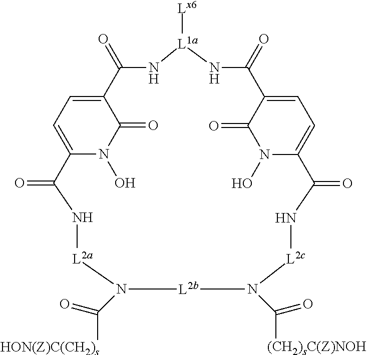

wherein the moieties are as defined herein above.

In some embodiments, the invention provides a chelating agent/metal ion chelate having the structure:

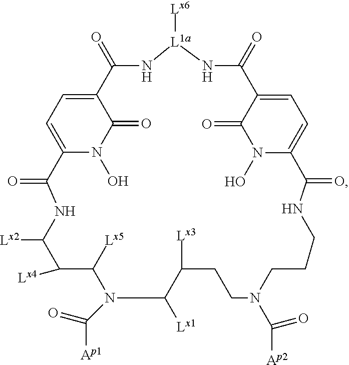

wherein L^{1a}, L^{x1}, L^{x2}, L^{x3}, L^{x4}, L^{x5}, and L^{x6} are linkers to a reactive functional group or to a targeting moiety as defined herein. Each of these linkers can be present or absent in a chelating agent/metal ion chelate of the invention. In an exemplary embodiment, at least one of L^{1a}, L^{x1}, L^{x2}, L^{x3}, L^{x4}, L^{x5} and L^{x6} is present. In an exemplary embodiment, at least L^{x6} is present. In an exemplary embodiment, at least two or at least three of these linkers are present. A^{b1}, A^{b2}, A^{p1}, and A^{p2} are as defined herein.

In some embodiments, the invention provides a chelating agent/metal ion chelate having the structure:

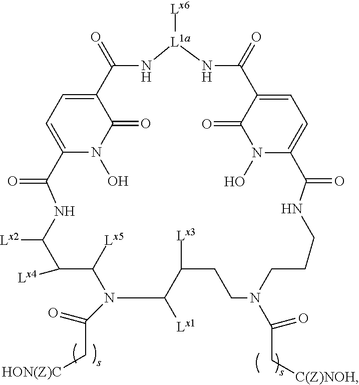

wherein L^{1a}, L^{x1}, L^{x2}, L^{x3}, L^{x4}, L^{x5}, and L^{x6} are linkers to a reactive functional group or to a targeting moiety as defined herein. Each of these linkers can be present or absent in a chelating agent/metal ion chelate of the invention. In an exemplary embodiment, at least one of L^{1a}, L^{x1}, L^{x2}, L^{x3}, L^{x4}, L^{x5}, and L^{x6} is present. In an exemplary embodiment, at least L^{x6} is present. In an exemplary embodiment, at least two or at least three of these linkers are present. A^{b1}, A^{b2}, A^{p1}, and A^{p2} are as defined herein. Z is O or S, and each index s is independently selected from the integers 1, 2, 3, 4, 5, and 6.

In the formulae immediately above, the bridging chelating moieties are displayed in an exemplary “up” orientation. The compounds of the invention also include chelating agents in which one or more of the bridging chelating moieties is in the “down” orientation.

Additional exemplary chelating agent/metal ion chelates are shown in the Examples.

### Deprotection

In various embodiments, the chelating agents of the invention are assembled using a protection/deprotection strategy to avoid participation of certain reactive residues in the reactions involved with cyclizing and elaborating the chelating agent. An exemplary protection/deprotection scheme is:

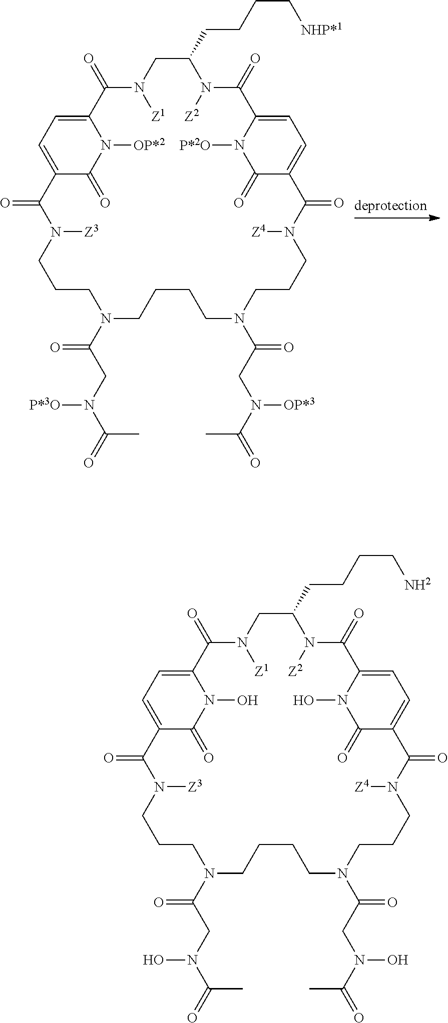

in which P*¹, P*², and P*³ represent generic protecting moieties. Conditions for placing and removing the protecting agents are well-known to those of ordinary skill in the art, and the synthetic literature is replete with methods for both placing and removing protection agents on and from structures. Exemplary protecting groups for amine linking arms (P*¹) as shown above are tert-butoxycarbonyl (Boc), benzyloxycarbonyl (Cbz), and fluorenylmethyloxycarbonyl (Fmoc). Exemplary protecting groups of use in assembling the bridging chelating agents of the invention (P*²) include benzyl protecting groups, para-methoxybenzyl (PMB), 2,4,6-trimethylbenzyl, triphenylmethyl (trityl), or tert-butyl (tBu). Exemplary protecting groups of use in assembling the pendant chelating agents of the invention (P*³) include benzyl protecting groups, para-methoxybenzyl (PMB), 2,4,6-trimethylbenzyl, triphenylmethyl (trityl), or tert-butyl (tBu). As those of skill in the art will appreciate, the above scheme is merely representative and any structure covered by the generic structures set forth herein can be supplanted for that shown above.

### Complexes

In one aspect, the invention provides a complex of a chelating agent disclosed herein with a metal ion.

Any of the combinations of chelating agents disclosed herein and a metal ion disclosed herein are encompassed by this disclosure and specifically provided by the invention.

In some embodiments, the metal ion chelate is luminescent.

Exemplary metal ion chelates are shown in the Examples.

In another aspect, the invention provides a metal ion chelate disclosed herein with an element, or ion thereof, from periods 4, 5, 6 and 7 and/or from groups 13, 14, 15, 16. In another aspect, the invention provides a metal ion chelate disclosed herein with an element, or ion thereof, from periods 3, 4, 7, 8, 9, 10, 11, 13, 14, and 15. In some embodiments, the invention provides a metal ion chelate disclosed herein with an element, or ion thereof, from periods 3, 4, and 13.

In some embodiments, chelating agents and metal ion complexes disclosed in WO 2013/187971 A2 are excluded.

### Metals

In some embodiments, the metal is an actinide. In some embodiments, the actinide is thorium (Th). In some embodiments, the metal is a lanthanide. In some embodiments, the lanthanide is terbium (Tb). In some embodiments, the lanthanide is europium (Eu). In some embodiments, the lanthanide is dysprosium (Dy). In some embodiments, the lanthanide is lutetium (Lu). In some embodiments, the lanthanide is gadolinium (Gd). In some embodiments the metal is yttrium (Y). In some embodiments, the metal is zirconium (Zr). In some embodiments, the metal ion is yttrium(III). In some embodiments, the metal ion is europium(III). In some embodiments, the metal ion is terbium(III). In some embodiments, the metal ion is zirconium(IV). In some embodiments, the metal ion is thorium(IV). In some embodiments, the metal ion is selected from Th⁴⁺, Zr⁴⁺, Eu³⁺, Dy³⁺, Tb³⁺, Lu³⁺, and Y³⁺. In some embodiments, the metal (ion) is a radionuclide. In some embodiments, the metal ion is ²²⁷Th(IV). In some embodiments, the metal ion is ⁸⁹Zr(IV).

In some embodiments, the metal is ¹⁷⁷Lu. In some embodiments, the metal is ¹⁶⁶Ho. In some embodiments, the metal is ¹⁵³Sm. In some embodiments, the metal is ⁹⁰Y. In some embodiments, the metal is ⁸⁶Y. In some embodiments, the metal is ¹⁶⁶Dy. In some embodiments, the metal is ¹⁶⁵Dy. In some embodiments, the metal is ¹⁶⁹Er. In some embodiments, the metal is ¹⁷⁵Yb. In some embodiments, the metal is ²²⁵Ac. In some embodiments, the metal is ¹⁴⁹Tb. In some embodiments, the metal is ¹⁵³Gd. In some embodiments, the metal is ²³⁰U.

In some embodiments, the metal is ¹¹¹In. In some embodiments, the metal is ⁶⁷Ga. In some embodiments, the metal is ⁶⁷Cu. In some embodiments, the metal is ⁶⁴Cu. In some embodiments, the metal is ¹⁶Re. In some embodiments, the metal is ¹⁸⁸Re. In some embodiments, the metal is ¹¹¹Ag. In some embodiments, the metal is ¹⁰⁹Pd. In some embodiments, the metal is ²¹²Pb. In some embodiments, the metal is ²⁰³Pb. In some embodiments, the metal is ²¹²Bi. In some embodiments, the metal is ²¹³Bi. In some embodiments, the metal is ^{195m}Pt. In some embodiments, the metal is ²⁰¹Tl. In some embodiments, the metal is ⁵⁵Co. In some embodiments, the metal is ^{99m}Tc.

In some embodiments, the metal is selected from yttrium (Y), a lanthanoid, an actinoid, zirconium (Zr), iron (Fe), and indium (In). In some embodiments, the metal is selected from zirconium (Zr), iron (Fe), indium (In), europium (Eu), holmium (Ho), lutetium (Lu), yttrium (Y), terbium (Tb), ytterbium (Yb), gadolinium (Gd), samarium (Sm), dysprosium (Dy), erbium (Er), and thorium (Th). In some embodiments, the metal is selected from Eu, Tb, Sm, and Dy. In some embodiments, the metal is Gd.

In some embodiments, the metal ion is selected from Zr(IV), Fe(III), Ga(III), In(III), Eu(III), Ho(III), Lu(III), Y(III), Sc(III), Tb(III), Yb(III), Gd(III), Sm(III), Dy(III), Er(III), and Th(IV). In some embodiments, the metal ion is selected from ²²⁷Th(IV), ⁸⁹Zr(IV), and ¹⁷⁷Lu(III).

In some embodiments, the metal is a radionuclide.

### Radionuclides

The chelating agents disclosed herein can be used to bind metal ions, in an exemplary embodiment, a radionuclide. The term “radionuclide” or “radioisotope” refers to a radioactive isotope or element with an unstable nucleus that tends to undergo radioactive decay. Numerous decay modes are known in the art and include alpha decay, proton emission, neutron emission, double proton emission, spontaneous fission, cluster decay, β⁻ decay, positron emission (β⁺ decay), electron capture, bound state beta decay, double beta decay, double electron capture, electron capture with positron emission, double positron emission, isomeric transition and internal conversion.

Exemplary radionuclides include alpha-emitters, which emit alpha particles during decay. In some embodiments, a radionuclide is an emitter of a gamma ray or a particle selected from an alpha particle, an electron and a positron.

In some embodiments, the radionuclide is an actinide. In some embodiments, the radionuclide is a lanthanide. In some embodiments, the radionuclide is a 3⁺ ion. In some embodiments, the radionuclide is a 4⁺ ion. In some embodiments the radionuclide is a 2⁺ ion.

Of particular use in exemplary metal ion chelates provided herein are radionuclides selected from isotopes of U, Pu, Fe, Cu, Sm, Gd, Tb, Dy, Ho, Er, Yb, Lu, Y, Th, Zr, In, Ga, Bi, Ra, At and Ac. In some embodiments, a radionuclide is selected form radium-223, thorium-227, astatine-211, bismuth-213, Lutetium-177, and actinium-225. Other useful radioisotopes include bismuth-212, iodine-123, copper-64, iridium-192, osmium-194, rhodium-105, samarium-153, and yttrium-88, yttrium-90, and yttrium-91. In exemplary embodiments, the radionuclide is thorium, particularly selected from thorium-227 and thorium-232. In some embodiments, thorium-226 is excluded. In some embodiments, U is excluded. In some embodiments, uranium-230 is excluded. That is, in some embodiments, a radionuclide is not U, or a radionuclide is not uranium-230 or a radionuclide is not thorium-226.

In a preferred embodiment, the radionuclide is selected from Th(IV)-227, Zr(IV)-89, Lu(III)-177, Y(III)-90, Y(III)-86, and In(III)-111.

In another preferred embodiment, the radionuclide is selected from Tb(III)-149, Tb(III)-161, Eu(III), Sm(III)-153 and Dy(III)-166.

In another preferred embodiment, the radionuclide is Ac(III)-225.

In some embodiments, the radionuclide is Tb(III)-149, Sc(III)-47, Dy(III)-166, Er(III)-169, Gd(III)-153, Ho(III)-166, Sm(III)-153, Yb(III)-175, Ac(III)-225, Bi(III)-212 or Bi(III)-213.

²³²Th exists in nature as an α-emitter with a half life of 1.4×10¹⁰ yr. In aqueous solution, Th(IV) is the only oxidation state. Thorium(IV) ion is bigger than Pu(IV) and usually forms complexes with 9 or higher coordination number. For example, the crystal structure of both Th(IV) complexes of simple bidentate 1,2-HOPO and Me-3,2-HOPO have been determined as nine coordinated species.

Similar to other actinide ions, thorium(IV) prefers forming complexes with oxygen, especially negative oxygen donor chelating moieties. Thorium(IV) also prefers octadentate or higher multidentate chelating moieties:

| Ligand | Acac | NTA | HEDTA* | EDTA** | DTPA | TTHA |
|---|---|---|---|---|---|---|
| Ligand | Bi-dentate | Tetra- | Hexa- | Hexa- | Octa- | Deca- |
| Type | | | | | | |
| Log K₁ | 7.85 | 16.9 | 18.5 | 25.3 | 30.34 | 31.9 |

*with one alcoholic oxygen and three carboxyl groups;
**with four carboxyl groups.

Other radionuclides with diagnostic and therapeutic value that can be used with the compounds disclosed herein can be found, for example, in U.S. Pat. Nos. 5,482,698 and 5,601,800; and Boswell and Brechbiel, Nuclear Medicine and Biology, 2007 October, 34(7): 757-778 and the manuscript thereof made available in PMC 2008 Oct. 1.

### Uses

The chelating agent/metal ion chelate disclosed herein can be used in a wide variety of therapeutic and diagnostic settings.

In one aspect, the invention provides a method of treating a disease in subject comprising administering a chelating agent/metal ion chelate disclosed herein to the subject, whereby the disease is ameliorated or eliminated.

In one aspect, the invention provides a method of diagnosing a disease in a subject comprising (a) administering a chelating agent/metal ion chelate disclosed herein to the animal and (b) detecting the presence or absence of a signal emitted by the chelating agent/metal ion chelate. In some embodiments, the detecting step comprises obtaining one or more images based on the signal.

In some embodiments, the disease is a hyperproliferative disorder, e.g., cancer.

In some embodiments, the chelating agent/metal ion chelate comprises a linker to a targeting moiety and the method further comprises localizing the chelating agent/metal ion chelateto a targeting site in the subject by binding the targeting moiety to the targeting site.

The chelating agent/metal ion chelate disclosed herein are particularly well suited for the preparation of stable, pre-labeled antibodies for use in the diagnosis and treatment of cancer and other diseases. For example, antibodies expressing affinity for specific tumors or tumor-associated antigens (e.g., melanoma, colon cancer, breast cancer, prostate cancer, etc.) are labeled with a diagnostic radionuclide-complexed chelate of this disclosure. Such antibodies are known in the art and are readily available. Where a metal ion chelate is used, it generally is covalently attached to the antibody. The antibodies used can be polyclonal or monoclonal, and the radionuclide-labeled antibodies can be prepared according to methods known in the art. The method of preparation will depend upon the type of radionuclide and antibody used. In various embodiments, the labeled antibodies are further stabilized through lyophilization. A stable, lyophilized, chelate-labeled antibody can be reconstituted with suitable diluent at the time of intended use, and a radionuclide added appropriate for the intended use, thus greatly simplifying the on-site preparation process.

The following examples are intended to illustrate certain embodiments of the invention and are not intended to be limiting of the scope of the invention in any way.

### EXAMPLES

The chelating agents/metal ion chelates of the invention are synthesized by an appropriate combination of generally well-known synthetic methods. Techniques useful in synthesizing the compounds of the invention are both readily apparent and accessible to those of skill in the relevant art. The discussion below is offered to illustrate certain of the diverse methods available for use in assembling the compounds of the invention, but it is not intended to limit the scope of reactions or reaction sequences that are useful in preparing the compounds of the present invention.

### Example 1. Synthesis of a Mono-Macrocyclic Bifunctional Chelator (Scheme 1)

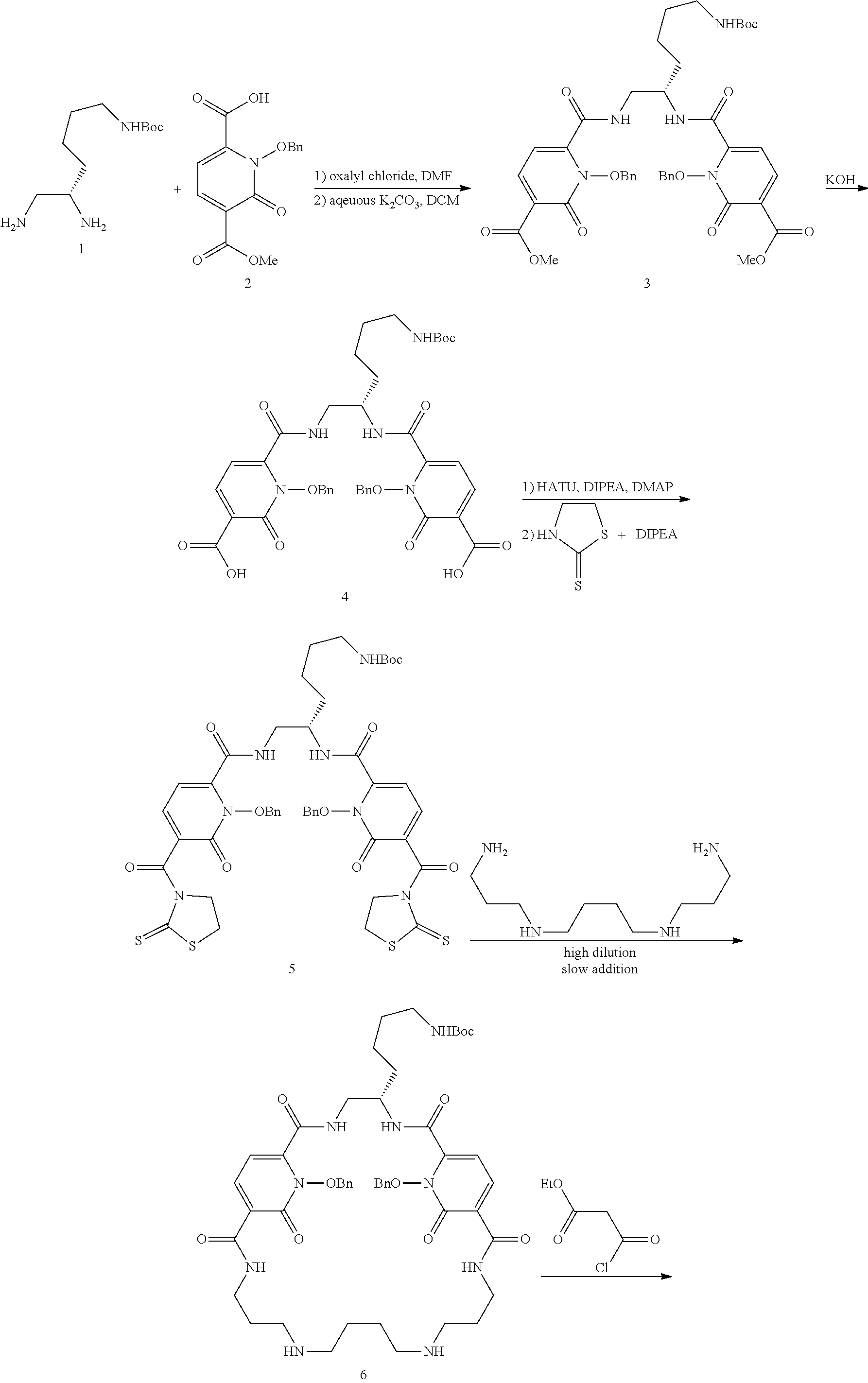

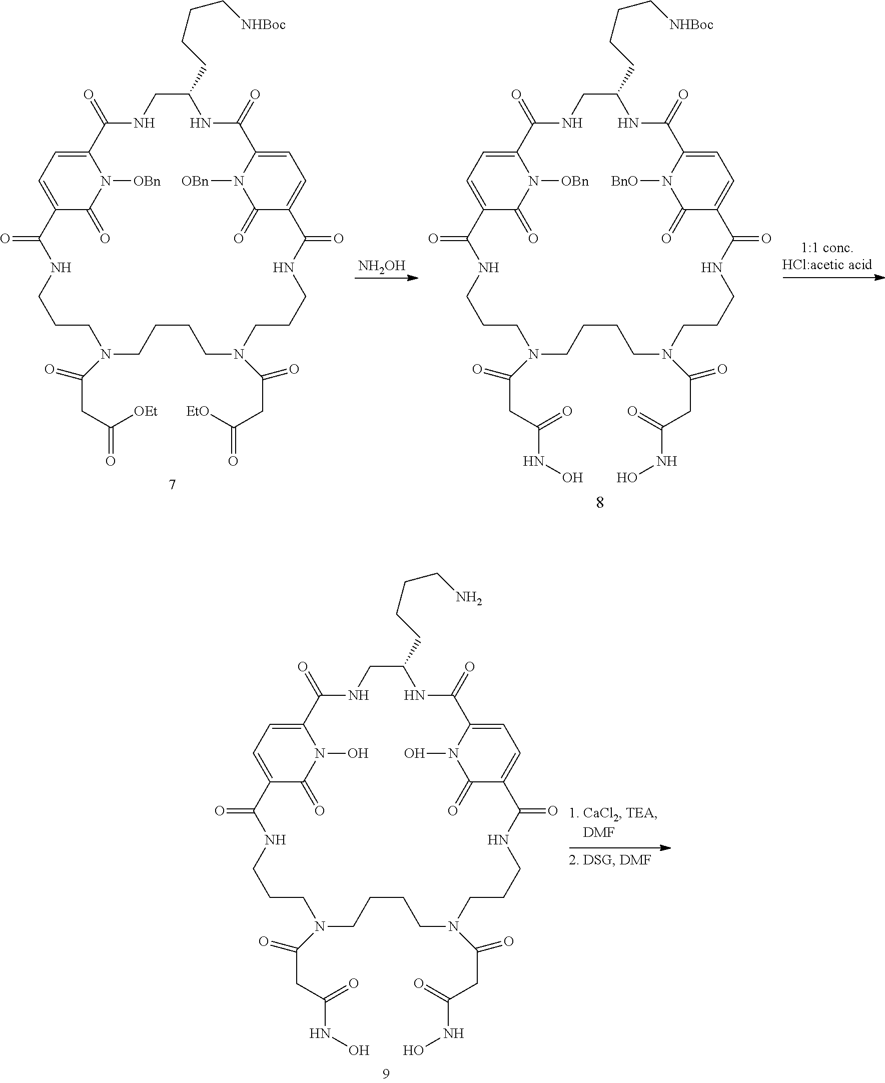

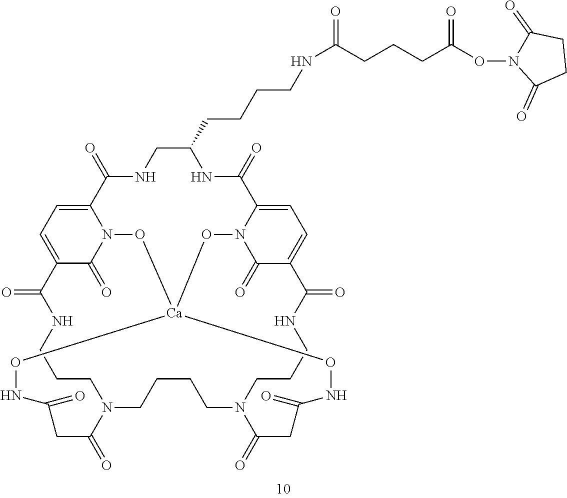

Diethyl 3,3′-((S,8Z,11Z)-9,30-bis(benzyloxy)-4-(4-((tert-butoxycarbonyl)amino)butyl)-2,7,10,12,27,29-hexaoxo-3,6,9,13,17,22,26,30-octaazatricyclo[26.2.2.2^{8,11}]tetratriaconta-1(31),8(34),11(33),28(32)-tetraene-17,22-diyl)bis(3-oxopropanoate) 7. To a solution of tert-butyl (4-((S,8Z,11Z)-9,30-bis(benzyloxy)-2,7,10,12,27,29-hexaoxo-3,6,9,13,17,22,26,30-octaazatricyclo[26.2.2.2^{81,11}]tetratriaconta-1(31),8(34),11(33),28(32)-tetraen-4-yl)butyl)carbamate 6 (Tatum D, Xu J, Magda D, Butlin N. Macrocyclic ligands with pendant chelating moieties and complexes thereof, WO/2019/173639) in dichloromethane is added ethylmalonyl chloride (two molar equivalents). Once the reaction is judged to be complete using HPLC, solvent is removed under reduced pressure and the product 7 is purified by silica gel chromatography using methanol in dichloromethane.

tert-Butyl (4-((S,8Z,11Z)-9,30-bis(benzyloxy)-17,22-bis(3-(hydroxyamino)-3-oxopropanoyl)-2,7,10,12,27,29-hexaoxo-3,6,9,13,17,22,26,30-octaazatricyclo[26.2.2.2^{8,11}]tetratriaconta-1(31),8(34),11(33),28(32)-tetraen-4-yl)butyl)carbamate 8. To a solution of compound 7 in ethanol is added an excess amount of hydroxylamine hydrochloride and triethylamine. The solution is heated. Once the reaction is judged to be complete using HPLC, solvent is removed under reduced pressure and the residue is dissolved in dichloromethane. After washing with water, solvent is removed under reduced pressure and the product 8 is used in the next step without further purification.

3,3′-((S,8Z,11Z)-4-(4-Aminobutyl)-9,30-dihydroxy-2,7,10,12,27,29-hexaoxo-3,6,9,13,17,22,26,30-octaazatricyclo[26.2.2.2^{8,11}]tetratriaconta-1(31),8(34),11(33),28(32)-tetraene-17,22-diyl)bis(N-hydroxy-3-oxopropanamide) 9. Compound 8 is dissolved in acetic acid and hydrochloric acid (12N) 1:1 v/v. The flask is sealed and allowed to stand for about 1 month. Hydrochloric acid is removed by a stream of air, then solvent is removed under reduced pressure and the crude product is dried in vacuo. The residue is dissolved in methanol and diethyl ether is added to form a precipitate. The precipitate is filtered, washed with diethyl ether, and dried in vacuo to provide crude product 9.

The calcium(II) complex of 2,5-dioxopyrrolidin-1-yl 5-((4-((S,8Z,11Z)-9,30-dihydroxy-17,22-bis(3-(hydroxyamino)-3-oxopropanoyl)-2,7,10,12,27,29-hexaoxo-3,6,9,13,17,22,26,30-octaazatricyclo[26.2.2.2^{8,11}]tetratriaconta-1(31),8(34),11(33),28(32)-tetraen-4-yl)butyl)amino)-5-oxopentanoate 10. Compound 9 is dissolved in dimethylformamide. A solution of calcium chloride in dimethylformamide (one molar equivalent) is added, and a solution of disuccinimidyl glutarate in dimethylformamide and triethylamine are added sequentially. The suspension is shaken until the reaction is judged to be complete using HPLC, whereupon the reaction mixture is partitioned into 2-mL microcentrifuge tubes. Diethyl ether (1.5 mL) is added to each tube, the contents mixed, and centrifuged at 12,000 rpm at once for 3 minutes. The supernatants are removed, the residues are washed with diethyl ether, and the pellets are air dried briefly then dried in vacuo. Each pellet is dissolved in dimethylformamide (250 μL), diethyl ether (1 mL) is added, the resulting suspensions are centrifuged, and the pellets are washed with ethyl ether as above. This precipitation is repeated one more time, and then the pellets are dried in vacuo to provide compound 10.

### Example 2. Synthesis of a Mono-Macrocyclic Bifunctional Chelator (Scheme 2)

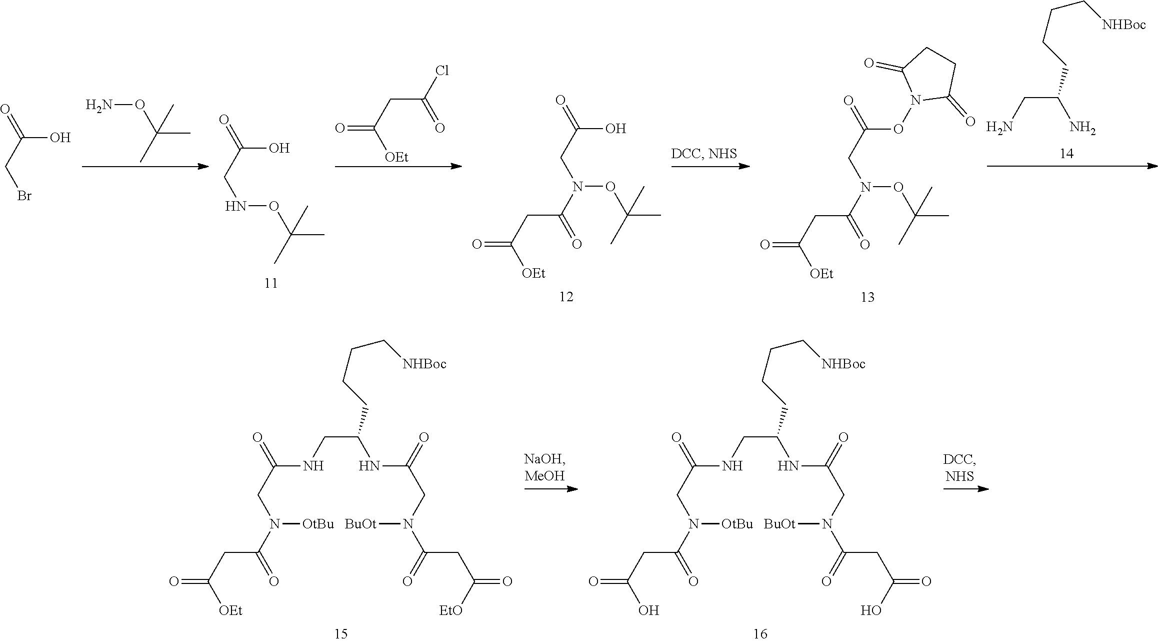

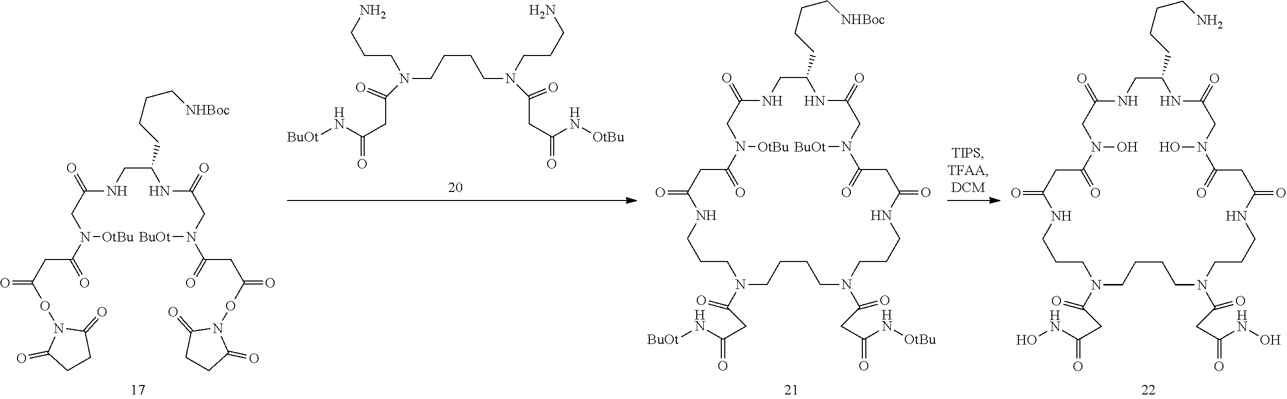

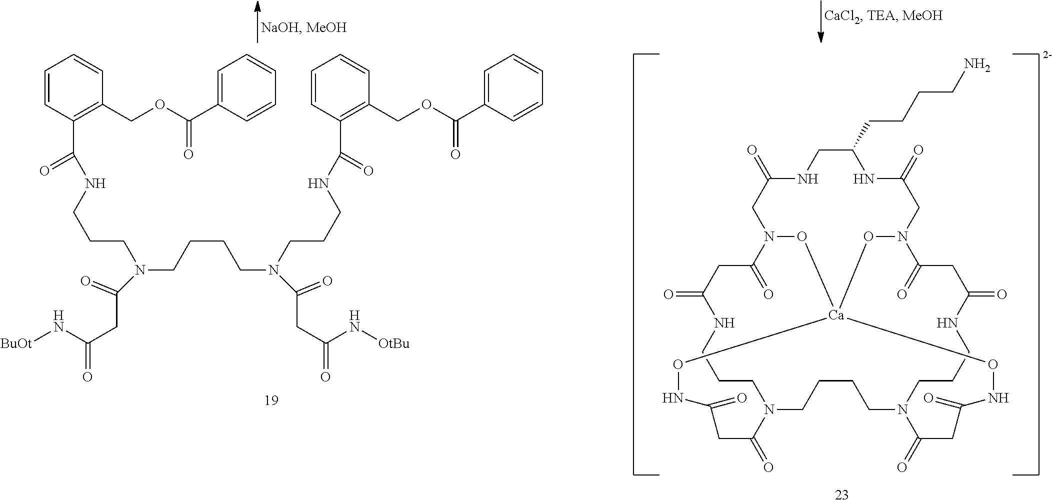

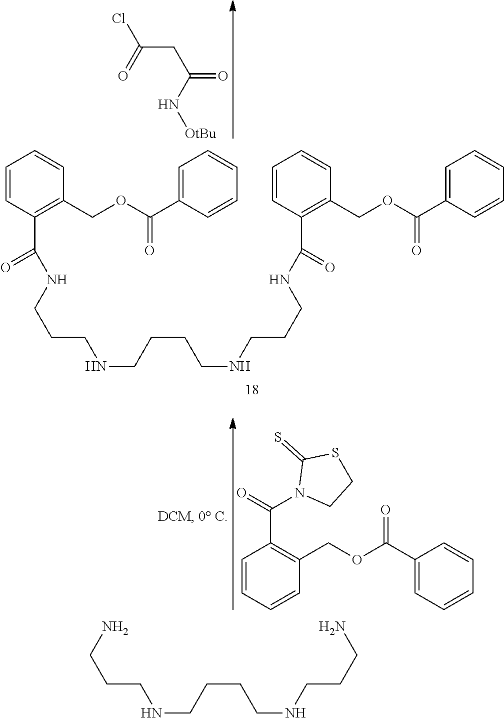

### Example 3. Synthesis of a Mono-Macrocyclic Bifunctional Chelator (Scheme 3)

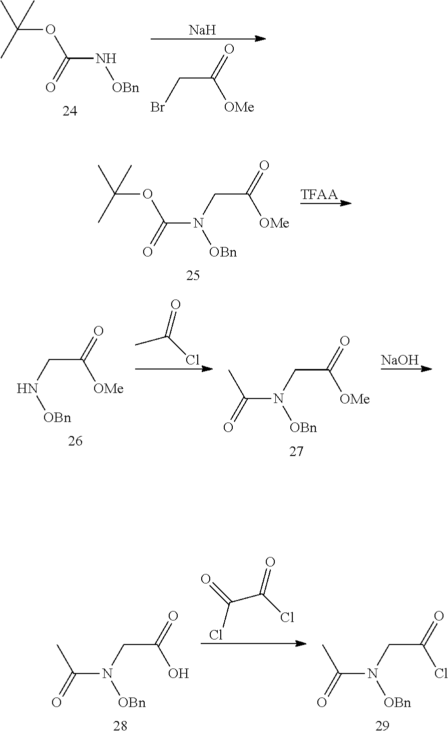

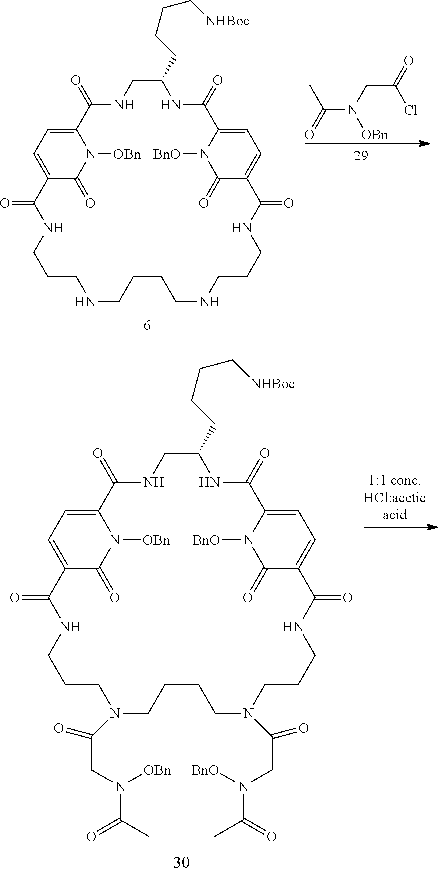 [SMILES: CC(C)(C)OC(=O)NCCCC[C@H]1CNC(=O)c2ccc(c(=O)n2OCc2ccccc2)C(=O)NCCCNCCCCNCCCNC(=O)c2ccc(n(OCc3ccccc3)c2=O)C(=O)N1 ; CC(=O)N(CC(=O)Cl)OCc1ccccc1 ; CC(=O)N(CC(=O)N1CCCCN(C(=O)CN(OCc2ccccc2)C(C)=O)CCCNC(=O)c2ccc(n(OCc3ccccc3)c2=O)C(=O)N[C@@H](CCCCNC(=O)OC(C)(C)C)CNC(=O)c2ccc(c(=O)n2OCc2ccccc2)C(=O)NCCC1)OCc1ccccc1]

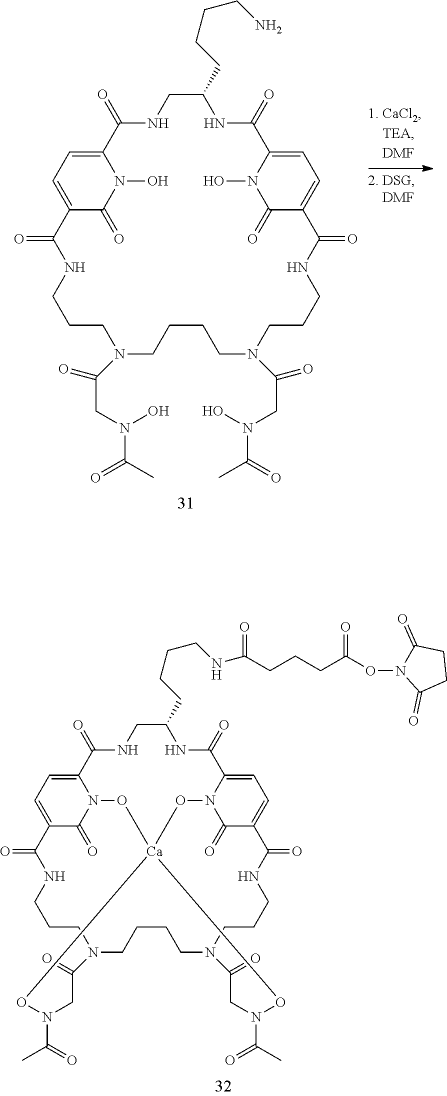

### Example 4. Synthesis of a Mono-Macrocyclic Bifunctional Chelator (Scheme 4)

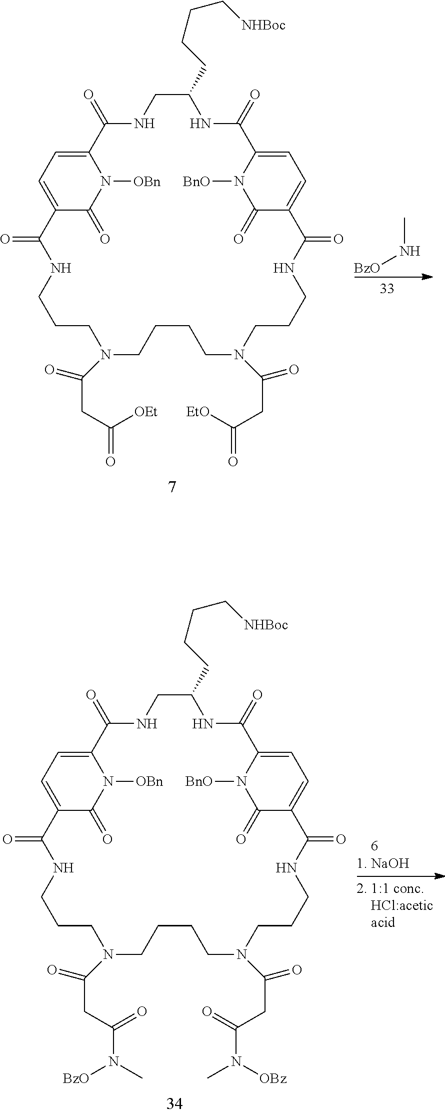

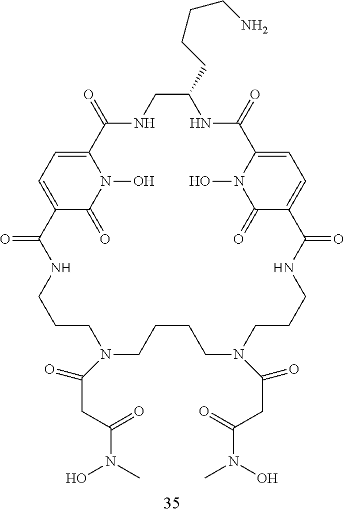 [SMILES: CN(O)C(=O)CC(=O)N1CCCCN(C(=O)CC(=O)N(C)O)CCCNC(=O)c2ccc(n(O)c2=O)C(=O)N[C@@H](CCCCN)CNC(=O)c2ccc(c(=O)n2O)C(=O)NCCC1]

### Example 5. Synthesis of a Mono-Macrocyclic Bifunctional Chelator (Scheme 5)

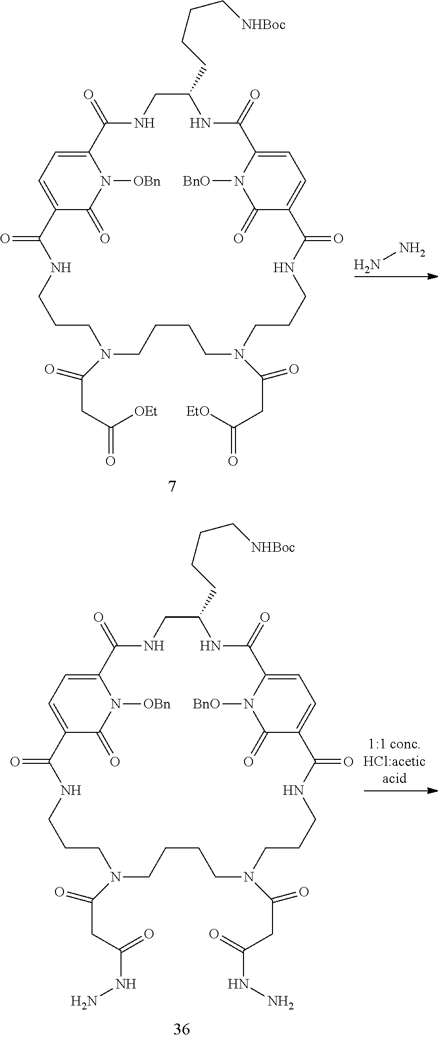

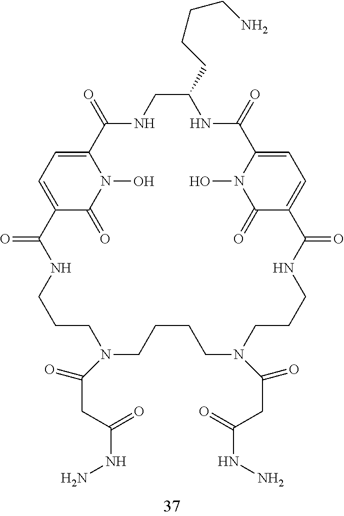 [SMILES: NCCCC[C@H]1CNC(=O)c2ccc(c(=O)n2O)C(=O)NCCCN(C(=O)CC(=O)NN)CCCCN(C(=O)CC(=O)NN)CCCNC(=O)c2ccc(n(O)c2=O)C(=O)N1]

### Example 6. Synthesis of a Mono-Macrocyclic Bifunctional Chelator (Scheme 6)

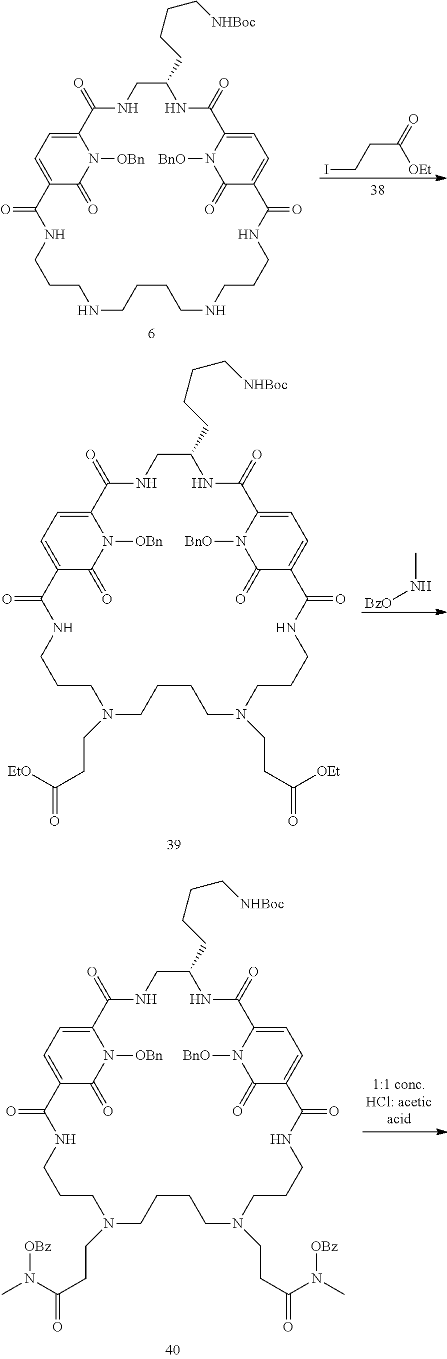

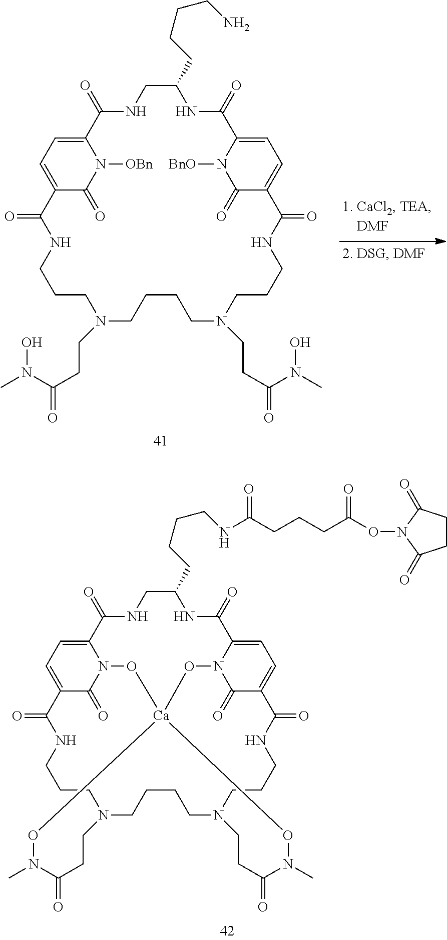

### Example 7. Synthesis of a Mono-Macrocyclic Bifunctional Chelator (Scheme 7)

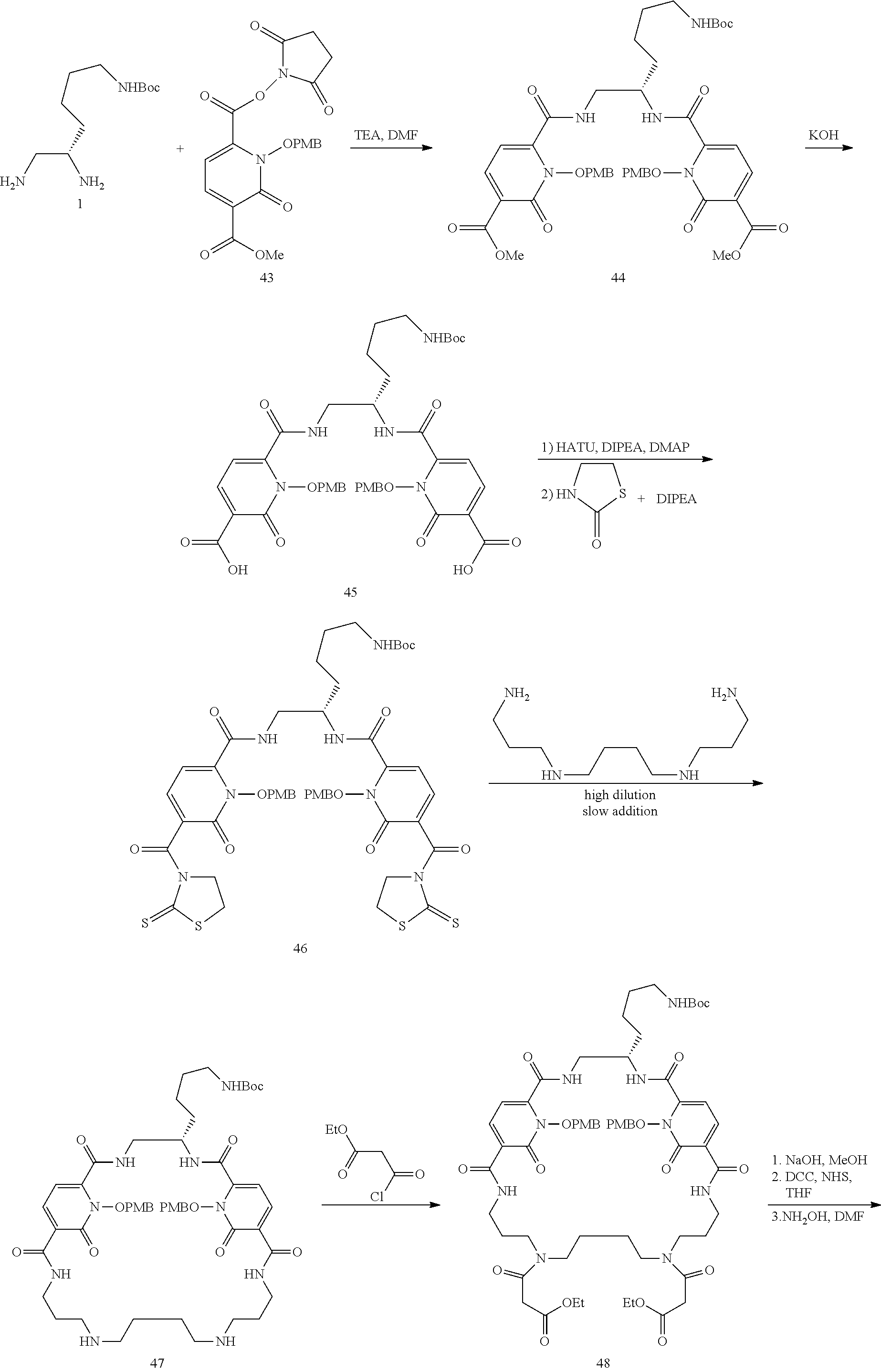

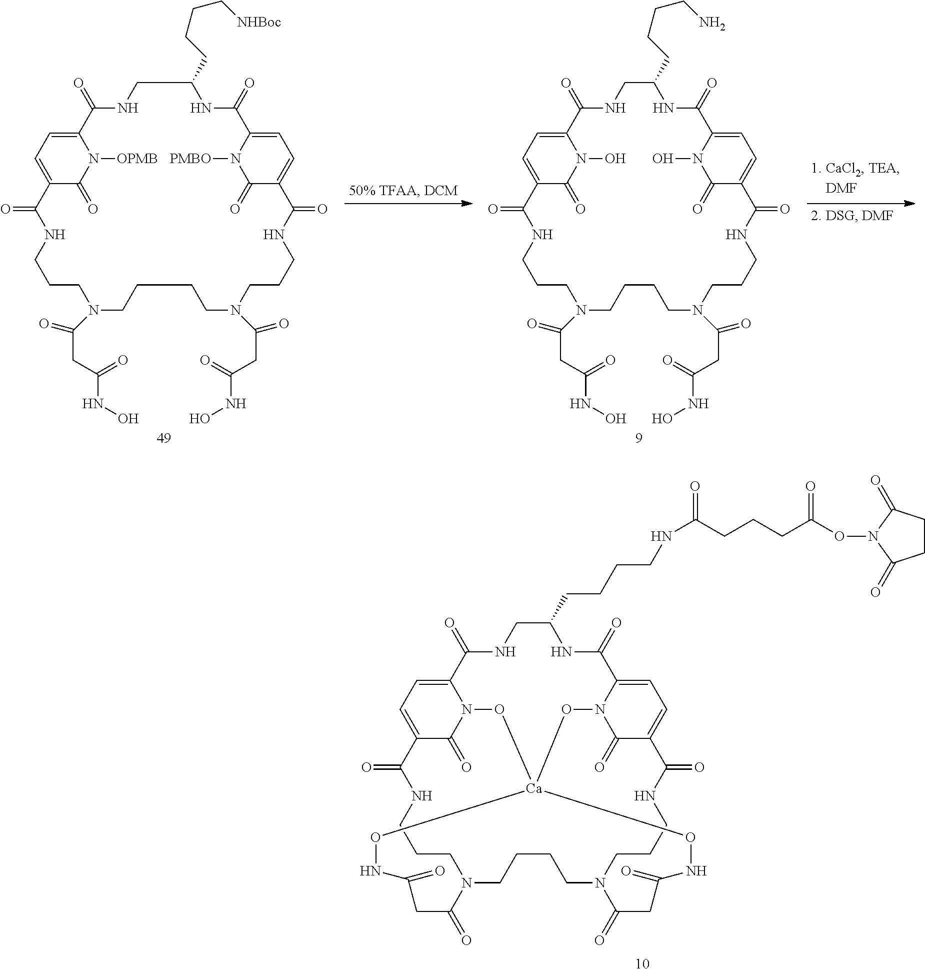

### Example 8. Synthesis of a Mono-Macrocyclic Bifunctional Chelator (Scheme 8)

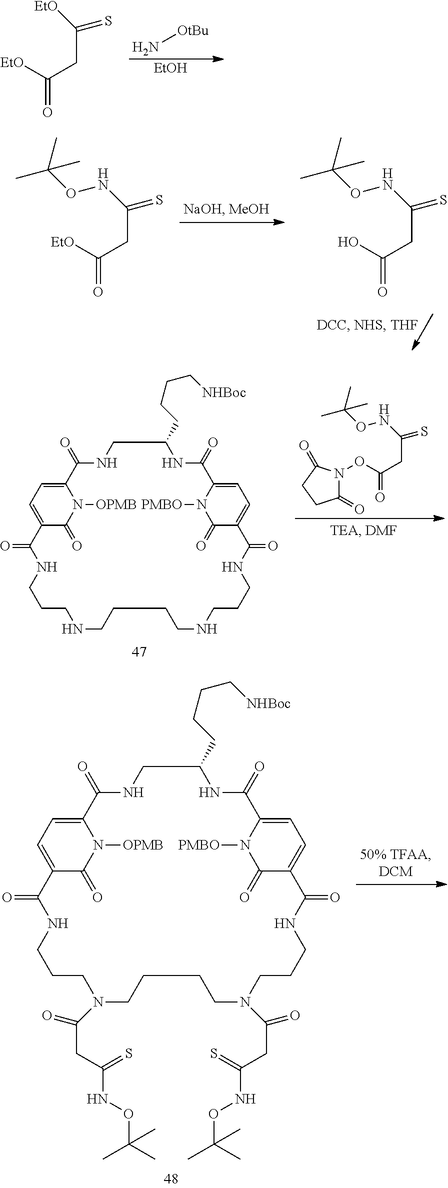

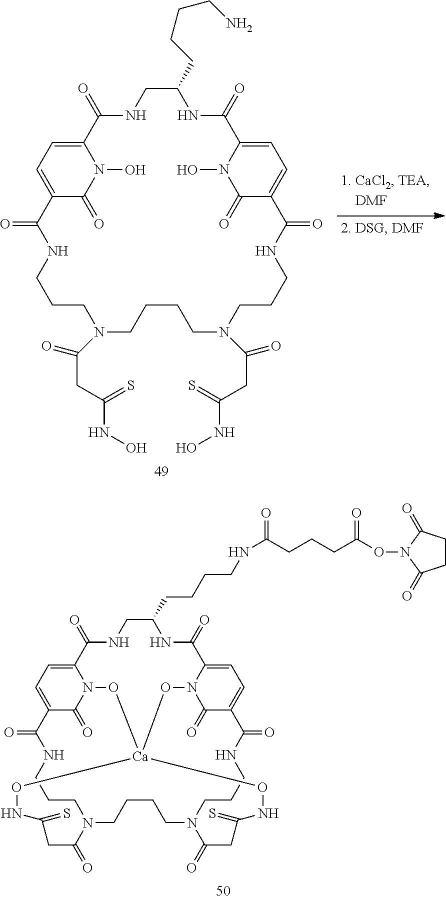

### Example 9. Synthesis of a Mono-Macrocyclic Bifunctional Chelator (Scheme 9)

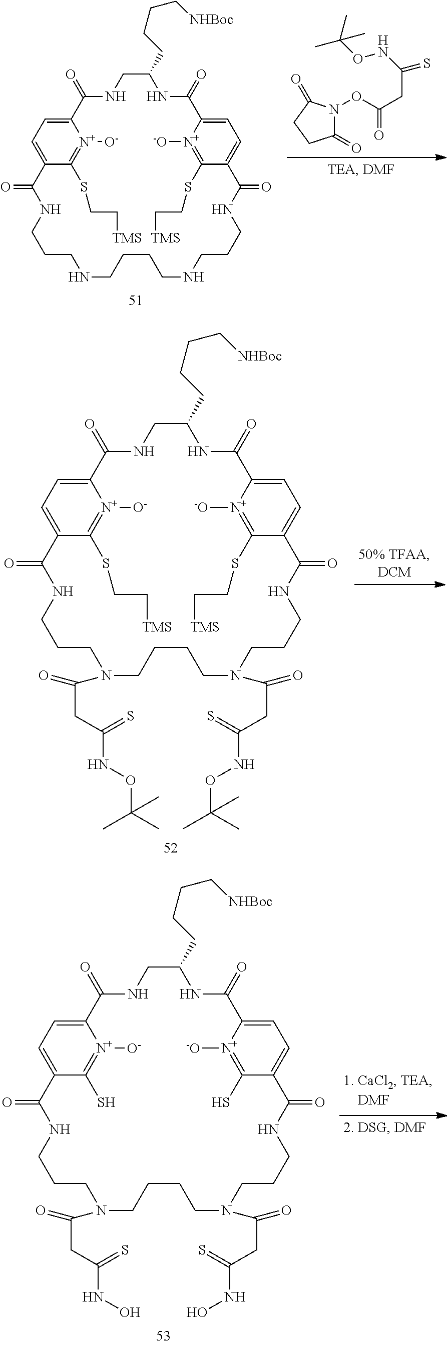

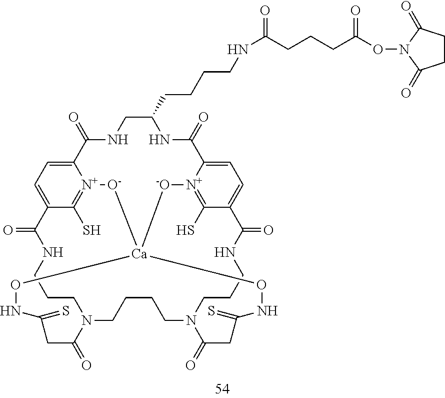

### Example 10. Synthesis of a Mono-Macrocyclic Bifunctional Chelator (Scheme 10)

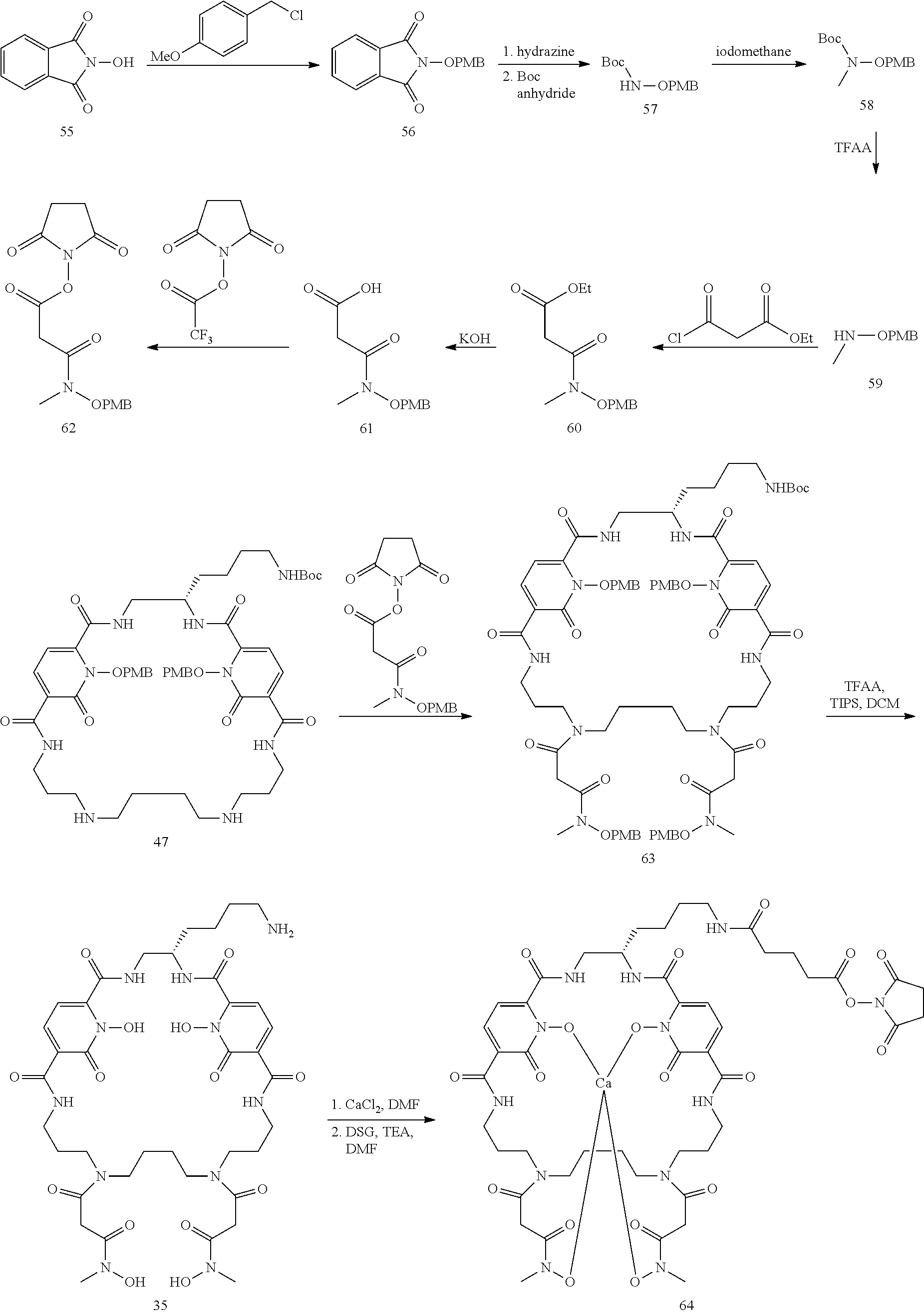

Compound 55 is commercially available. Compounds 56, 57, 58, and 59 have been previously reported in the literature (Wencewicz T, Yang B, Rudloff J, Oliver A, Miller M. J. Med. Chem. N—O Chemistry for Antibiotics: Discovery of N-Alkyl-N-(pyridin-2-yl)hydroxylamine Scaffolds as Selective Antibacterial Agents Using Nitroso Diels-Alder and Ene Chemistry, 2011, 54(19), 6843). Compound 62 is shown as the NHS ester, but other activation methods can also be used instead. For example, an acyl chloride of 61 can be generated from reaction with oxalyl chloride or thionyl chloride with DMF as a catalyst. Alternative activated esters (other than the N-hydroxysuccinimide ester shown) can also be generated, such as tetrafluorophenyl or p-nitrophenyl. One could also directly react 61 with 47 in a one-pot activation protocol using HATU, TBTU, or PyBOP in the presence of DMAP and DIPEA. These are just a few examples of the many ways generate activated esters and/or facilitate amide bond formation.

### Example 11. Synthesis of a Mono-Macrocyclic Bifunctional Chelator (Scheme 11)

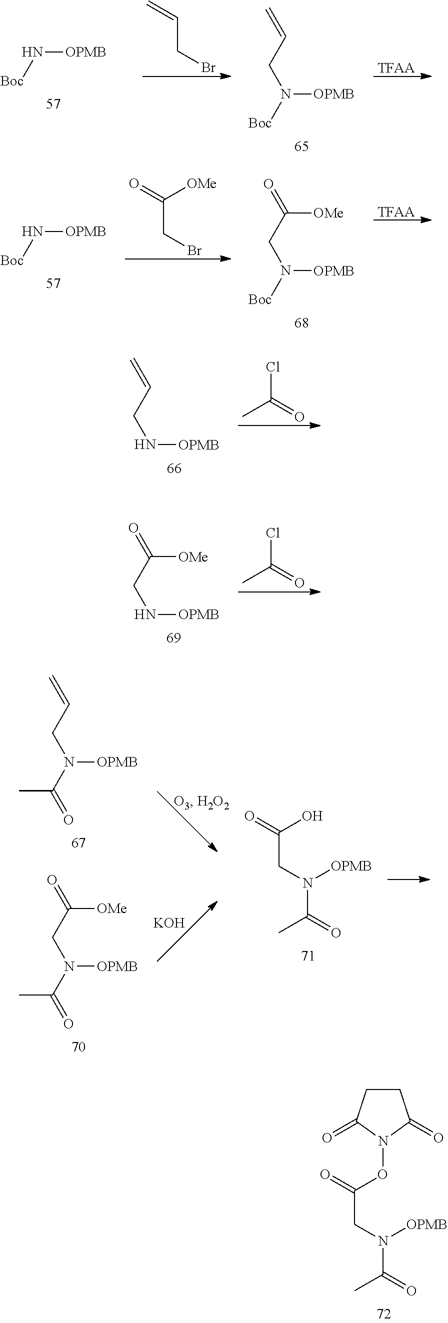

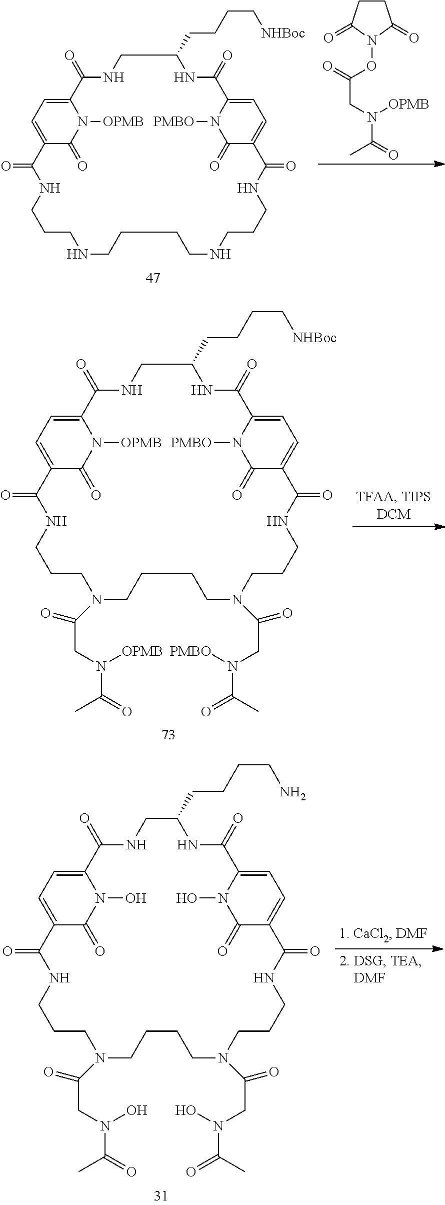

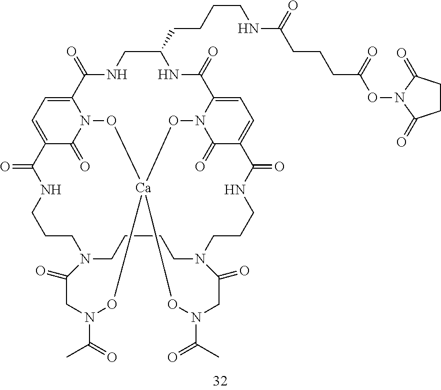

Compounds 65 and 66 have been previously reported in the literature (Wencewicz T, Yang B, Rudloff J, Oliver A, Miller M. J. Med. Chem. N—O Chemistry for Antibiotics: Discovery of N-Alkyl-N-(pyridin-2-yl)hydroxylamine Scaffolds as Selective Antibacterial Agents Using Nitroso Diels-Alder and Ene Chemistry, 2011, 54(19), 6843). Compound 72 is shown as the NHS ester, but other activation methods can also be used instead. For example, an acyl chloride of 71 can be generated from reaction with oxalyl chloride or thionyl chloride with DMF as a catalyst. Alternative activated esters (other than the N-hydroxysuccinimide ester shown) can also be generated, such as tetrafluorophenyl or p-nitrophenyl. One could also directly react 71 with 47 in a one-pot activation protocol using HATU, TBTU, or PyBOP in the presence of DMAP and DIPEA. These are just a few examples of the many ways generate activated esters and/or facilitate amide bond formation.

### Example 12. Synthesis of a Mono-Macrocyclic Bifunctional Chelator (Scheme 12)

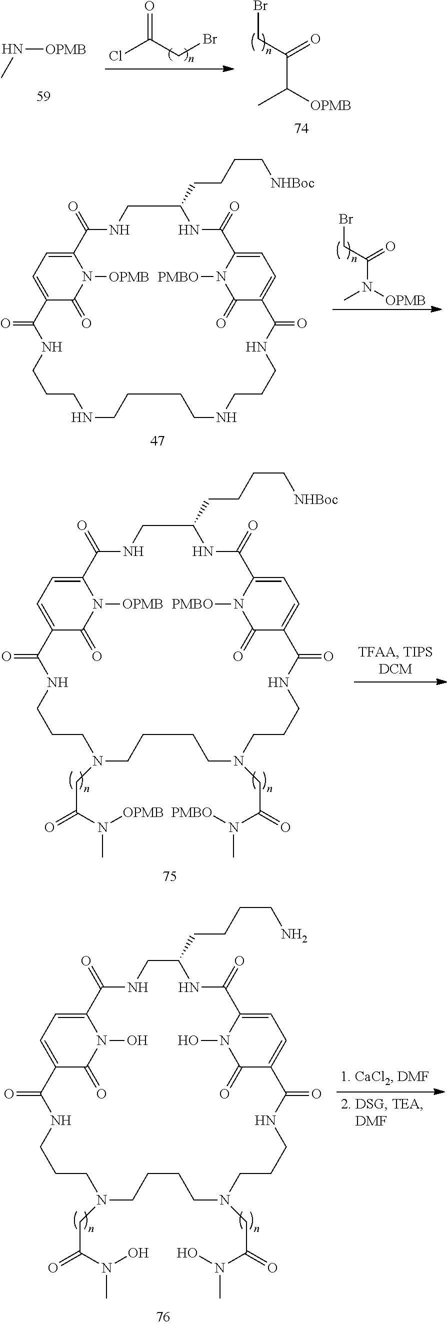

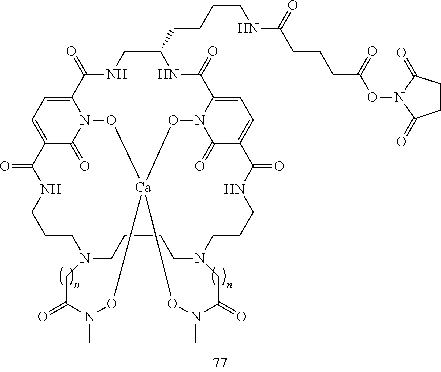

The index n can be 1, 2, 3, 4, 5, or 6.

### Example 13. Synthesis of a Mono-Macrocyclic Bifunctional Chelator (Scheme 13)

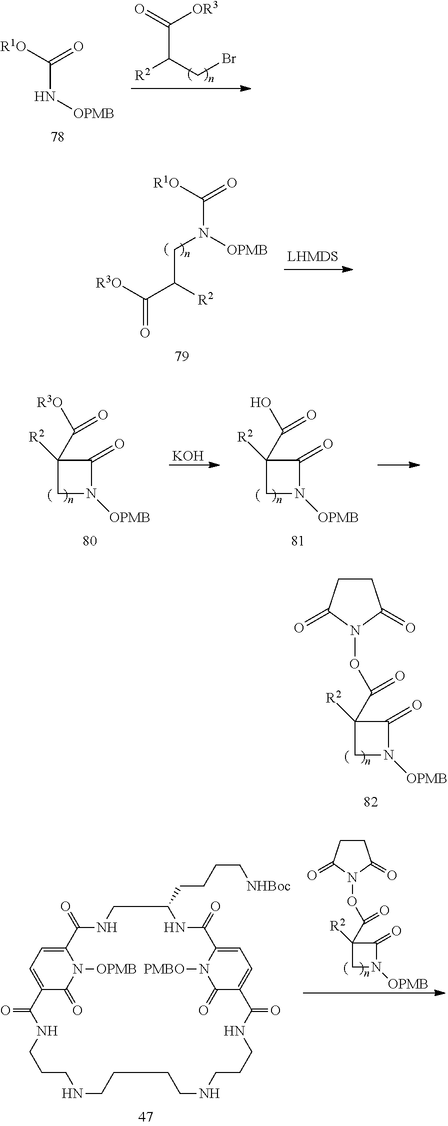

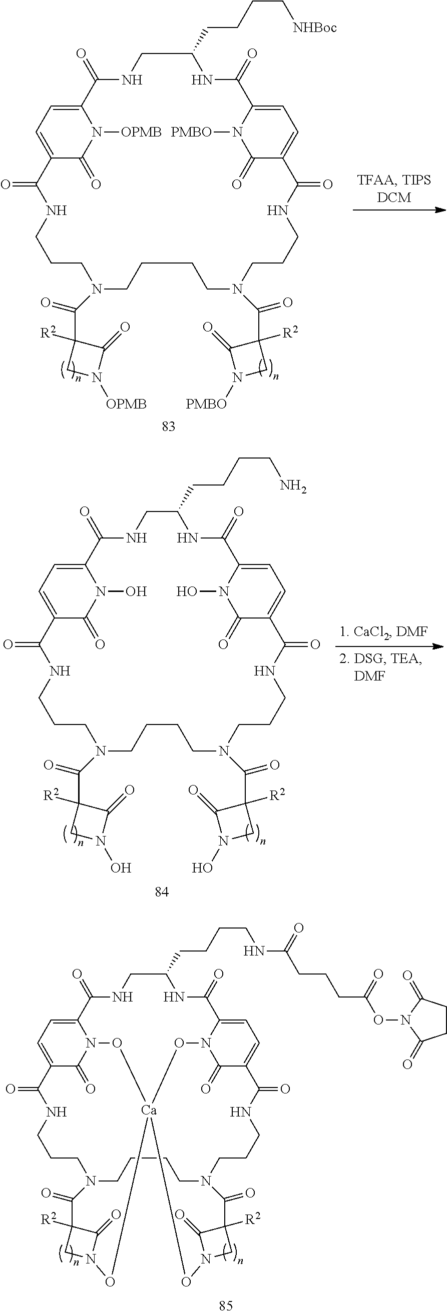

The index n can be 1, 2, 3, 4, 5, or 6. Benzyl protected (instead of PMB protected) analogs of 78, 79, 80, and 81 (where n=2 or 3) have been reported in the literature. Compound 82 is shown as the NHS ester, but other activation methods can also be used instead. For example, an acyl chloride of 81 can be generated from reaction with oxalyl chloride or thionyl chloride with DMF as a catalyst. Alternative activated esters (other than the N-hydroxysuccinimide ester shown) can also be generated, such as tetrafluorophenyl or p-nitrophenyl. One could also directly react 81 with 47 in a one-pot activation protocol using HATU, TBTU, or PyBOP in the presence of DMAP and DIPEA. These are just a few examples of the many ways generate activated esters and/or facilitate amide bond formation.

### Example 14. Synthesis of a Mono-Macrocyclic Bifunctional Chelator (Scheme 14)

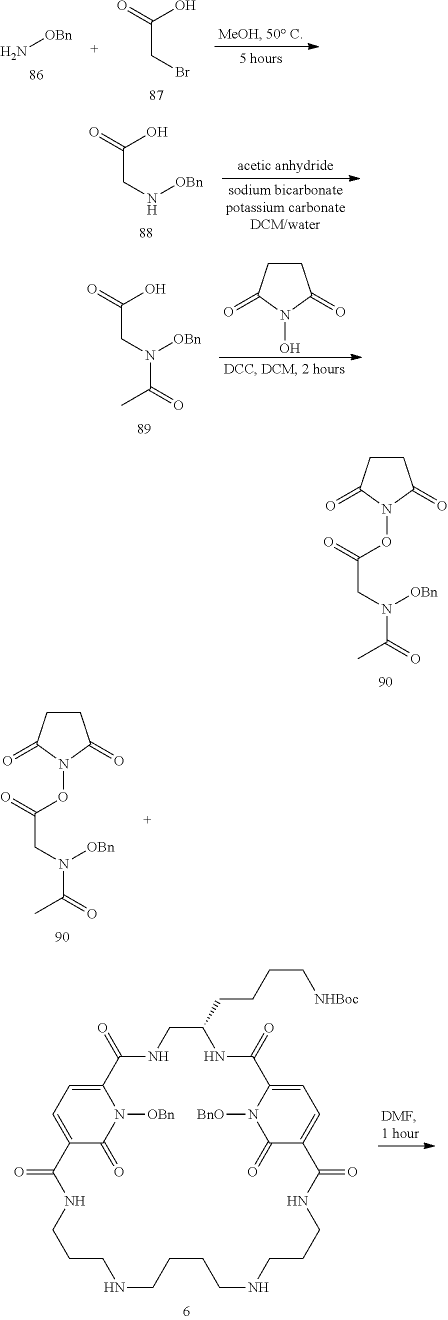

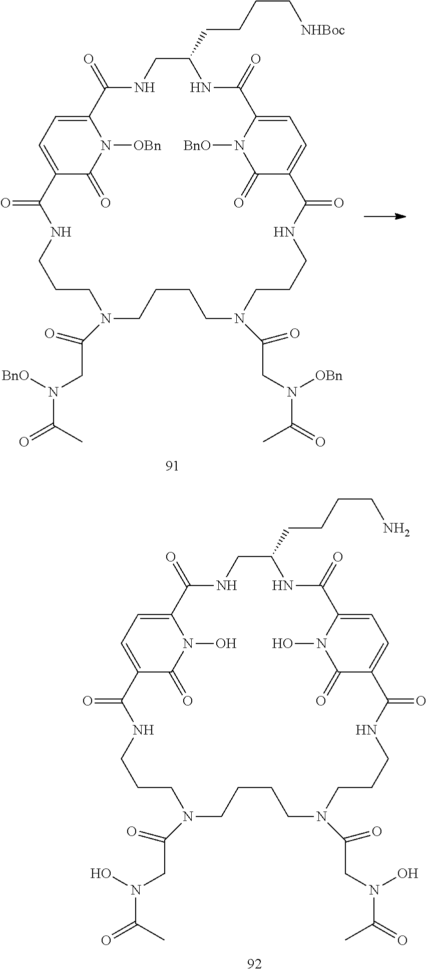 [SMILES: CC(=O)N(CC(=O)N1CCCCN(C(=O)CN(OCc2ccccc2)C(C)=O)CCCNC(=O)c2ccc(n(OCc3ccccc3)c2=O)C(=O)N[C@@H](CCCCNC(=O)OC(C)(C)C)CNC(=O)c2ccc(c(=O)n2OCc2ccccc2)C(=O)NCCC1)OCc1ccccc1 ; CC(=O)N(O)CC(=O)N1CCCCN(C(=O)CN(O)C(C)=O)CCCNC(=O)c2ccc(n(O)c2=O)C(=O)N[C@@H](CCCCN)CNC(=O)c2ccc(c(=O)n2O)C(=O)NCCC1]

The precursors O-benzylhydroxylamine hydrochloride (86 HCl, CAS: 2687-43-6) and bromoacetic acid (87, CAS: 79-08-3) were purchased from Chem-Impex (Wood Dale, IL). All other solvents and reagents were purchased from commercial sources and used as received unless otherwise noted. Compounds 88 and 89 were prepared according to procedures modified from previously reported methods (Kolasa T, Chimiak A. O-protected derivatives of N-hydroxyamino acids, Tetrahedron 1974, 30, 3591 and Inomata T, Eguchi H, Funahashi Y, Ozawa T, Masuda H. Adsorption Behavior of Microbes on a QCM Chip Modified with an Artificial Siderophore-Fe³⁺ Complex, Langmuir 2012, 28(2), 1611). High resolution electrospray ionization mass spectra (HRMS-ESI) were performed by the Microanalytical Laboratory at the University of California, Berkeley.

2-((Benzyloxy)amino)acetic acid (88). O-Benzylhydroxylamine hydrochloride (86 HCl, 40.2 g, 252 mmol) was added to water (150 mL) containing potassium hydroxide (14.1 g, 252 mmol), and the suspension was stirred for 30 minutes at room temperature. Next, potassium carbonate (10.0 g, 72.4 mmol) was added, followed by stirring for an additional 30 min at room temperature, during which time the desired freebase 86 separated from the aqueous phase in the form of an oil. The compound 86 (O-benzylhydroxylamine) was separated from the aqueous phase by extraction with dichloromethane (3×150 mL), and the dichloromethane solution was reduced by rotary evaporation and then high vacuum overnight to afford an oil of 86. The residue of 86 was dissolved into methanol (200 mL), and bromoacetic acid (87, 12.8 g, 84 mmol) was then added to the methanolic solution. The reaction was then heated to 50° C. with stirring for 5 hours. Upon cooling, the methanol was removed under vacuum by rotary evaporation. The residue was then dissolved in water (250 mL), and the excess O-benzylhydroxylamine was removed by extraction with dichloromethane (3×150 mL). The aqueous solution was then isolated and the final traces of dichloromethane were removed under vacuum by rotary evaporation. Hydrochloric acid was added dropwise to the aqueous solution until a significant precipitate was formed and the pH of the aqueous phase was found to be approximately neutral (pH ˜6) by pH indicator paper. The solid material was collected by filtration, washed with water (3×50 mL), and then dried under vacuum overnight to afford a free-flowing white powder of 88. Yield 5.34 g, 35%. HRMS-ESI (m z, [M−H]⁻) Calcd for C₉H₁₀NO₃: 180.0666, Found: 180.0667.

N-Acetyl-N-(benzyloxy)glycine (89). Potassium carbonate (2.47 g, 17.9 mmol) and sodium bicarbonate (7.52 g, 89.5 mmol) were dissolved in water (100 mL). The starting material 2-((benzyloxy)amino)acetic acid (88, 3.24 g, 17.9 mmol) was added to the aqueous solution, followed by dichloromethane (100 mL). Next, acetic anhydride (3.65 g, 35.8 mmol) was added dropwise with vigorous stirring at room temperature, and the reaction mixture was stirred at room temperature for 3 hours. Potassium hydroxide (2.01 g, 35.8 mmol) was then added to the reaction mixture to ensure complete consumption of the acetic anhydride reagent, and the reaction mixture was stirred at room temperature overnight. Upon completion, the stirring was stopped and the aqueous phase was separated and washed with dichloromethane (3×100 mL). The aqueous solution was then acidified by dropwise addition of hydrochloric acid until the evolution of carbon dioxide ceased and the solution was found to be acidic by pH paper. The desired product was then extracted from the aqueous phase by extraction with dichloromethane (3×150 mL). The dichloromethane solution was reduced by rotary evaporation and then by high vacuum overnight to afford a solid of 89. The solid was mechanically pulverized and the final traces of acetic acid were removed under high vacuum overnight to afford a dense, free-flowing white powder of 89. Yield 3.88 g, 97.1%. HRMS-ESI (m/z, [M−H]⁻) Calcd for C₁₁H12NO₄: 222.0772, Found: 222.0772.

2,5-Dioxopyrrolidin-1-yl 2-(N-(benzyloxy)acetamido)acetate (90). The starting material N-acetyl-N-(benzyloxy)glycine (89, 1.00 g, 4.48 mmol) was dissolved into dichloromethane (30 mL), N-hydroxysuccinimide (619 mg, 5.38 mmol) was added, and the suspension was stirred for 1 hour at room temperature. The reagent N,N′-dicyclohexylcarbodiimide (1.0 M in dichloromethane, 5.00 mL, 5.00 mmol) was then added dropwise, and the reaction mixture was stirred for 2 hours at room temperature. The reaction mixture was concentrated by rotary evaporation to approximately 10 mL in volume, and the N,N′-dicyclohexylurea byproduct was removed by filtration and washed with the dichloromethane (3×5 mL). The collected dichloromethane eluents were concentrated to approximately 5 mL in volume by rotary evaporation, and then the concentrated solution was added dropwise to diethyl ether (75 mL). The resulting suspension sealed with a cap and allowed to stand at room temperature overnight. The fluffy white solid of the desired product was collected by filtration, washed with diethyl ether (3×15 mL), and dried under high vacuum overnight to afford a free-flowing white powder of 90. Yield 1.14 g, 79.4%. HRMS-ESI (m z, [M+Na]*) Calcd for C₁₅H₁₆N₂O₆Na: 343.0901, Found: 343.0904.

tert-Butyl (4-((S,8Z,11Z)-17,22-bis(N-acetyl-N-(benzyloxy)glycyl)-9,30-bis(benzyloxy)-2,7,10,12,27,29-hexaoxo-3,6,9,13,17,22,26,30-octaazatricyclo[26.2.2.2^{8,11}]tetratriaconta-1(31),8(34),11(33),28(32)-tetraen-4-yl)butyl)carbamate (91). The starting material (S)-tert-butyl (4-(9,30-bis(benzyloxy)-2,7,10,12,27,29-hexaoxo-3,6,9,13,17,22,26,30-octaazatricyclo[26.2.2.2^{8,11}]tetratriaconta-1(31),8(34),11(33),28(32)-tetraen-4-yl)butyl)carbamate (6) was prepared as described previously (Tatum D, Xu J, Magda, D, Butlin N. Macrocyclic ligands with pendant chelating moieties and complexes thereof, WO/2019/173639). The crude preparation of 6 (200 mg) was dissolved in dichloromethane (25 mL). The 2-mercaptothiazoline byproduct generated from the preparation of 6 was removed by aqueous extraction using 0.5 M potassium hydroxide (3×10 mL), and then traces of potassium hydroxide were removed by extraction with water (3×10 mL). The dichloromethane solution of 6 was concentrated under vacuum by rotary evaporation and then high vacuum overnight to afford a hardened foam of 6. A portion of the hardened foam of 6 (20.6 mg, 21.9 mol) was dissolved into N,N-dimethylformamide (600 L), and then 2,5-dioxopyrrolidin-1-yl 2-(N-(benzyloxy)acetamido)acetate (90, 35.1 mg, 110 mol) was added. The reaction proceeded at room temperature for 1 hour. The desired product was isolated by reverse-phase (Agilent Eclipse XDB-C18, 5 μm, 4.6×150 mm) HPLC (1 mL injection loop) in 100 μL increments with a 10% to 60% acetonitrile in 0.1% aqueous TFA gradient (0 to 20 min, 1 mL/min) followed by 80% acetonitrile (20.5 to 24.5 min, 1 mL/min) elution, with the desired product eluting at approximately 23 min. The eluents collected from the HPLC were concentrated under vacuum by rotary evaporation and then by high vacuum overnight to afford a residue of 91. Yield 3.4 mg, 11.5%. HRMS-ESI (m z, [M+H]+) Calcd for C₇H₈₈N₁₁O₁₆: 1350.6405, Found: 1350.6387.

N,N′—(((S,8Z,11Z)-4-(4-aminobutyl)-9,30-dihydroxy-2,7,10,12,27,29-hexaoxo-3,6,9,13,17,22,26,30-octaazatricyclo[26.2.2.2^{8,11}]tetratriaconta-1(31),8(34),11(33),28(32)-tetraene-17,22-diyl)bis(2-oxoethane-2,1-diyl))bis(N-hydroxyacetamide) (92). The starting material tert-butyl (4-((S,8Z,11Z)-17,22-bis(N-acetyl-N-(benzyloxy)glycyl)-9,30-bis(benzyloxy)-2,7,10,12,27,29-hexaoxo-3,6,9,13,17,22,26,30-octaazatricyclo[26.2.2.2^{8,11}]tetratriaconta-1(31),8(34),11(33),28(32)-tetraen-4-yl)butyl)carbamate (91) is deprotected by treatment with HBr in acetic acid, or by treatment with BCl₃ in dichloromethane, or by treatment with BBr₃ in dichloromethane, or by treatment with boron trifluoroacetate in TFA, or by treatment with H₂ and Pd/C in methanol, or by treatment with H₂ and poisoned Pd/C in methanol, or by treatment with H₂ and Pd/C in methanol containing a metal salt (e.g. EuCl₃·6H₂O) followed by treatment with acid to remove the metal, or by treatment with HCl in acetic acid for one month followed by treatment with a metal salt (e.g. EuCl₃·6H₂O) in N,N-dimethylformamide followed by treatment with HCl to remove the metal, or any combination thereof. The synthesis of 92 is optionally improved by replacement of O-benzylhydroxylamine (86) with 0-(4-methoxybenzyl)hydroxylamine (CAS: 21038-22-2), 0-(2,4,6-trimethylbenzyl)hydroxylamine (CAS: 52245-11-1), O-(triphenylmethyl)hydroxylamine (CAS: 31938-11-1), or O-tert-butylhydroxylamine (CAS: 37477-16-0) in Scheme 14, and then proceeding in the aforementioned synthesis using the described procedures or modifications thereof. Analogous protecting substitutions may be made for any of the examples in this disclosure where benzyl or 4-methoxybenzyl (Bn or PMBO) has been specified. The benzyl protecting groups on the 1,2-HOPOs are also optionally replaced by 4-methoxybenzyl protecting groups (i.e. compound 6 is replaced with compound 47) as shown in examples 7, 8, 10, 11, 12, and 13. As above for the hydroxamate chelators, the benzyl protecting groups on the 1,2-HOPOs are optionally replaced by 2,4,6-trimethylbenzyl, triphenylmethyl (trityl), or tert-butyl to facilitate a more facile deprotection reaction.

### Example 15. Alternative Synthesis of Protected Bifunctional Chelator 91 (Scheme 15)

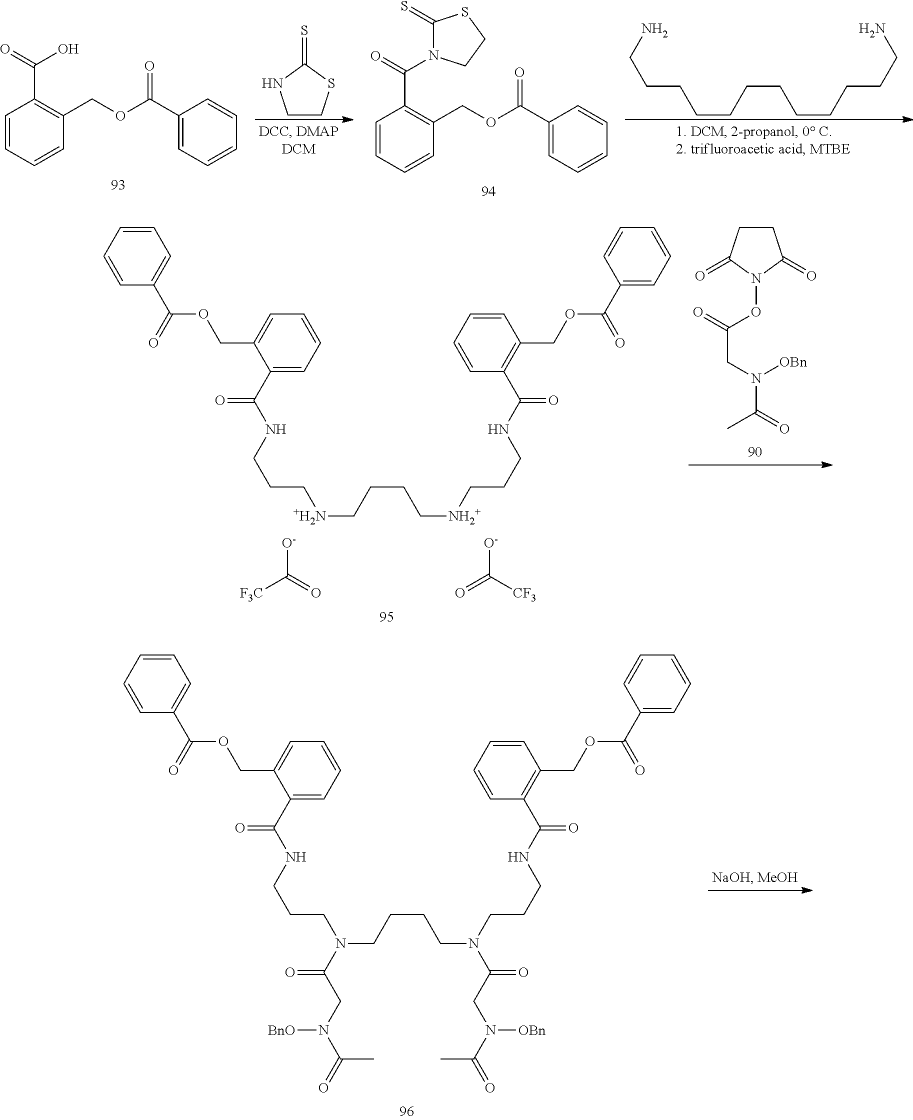

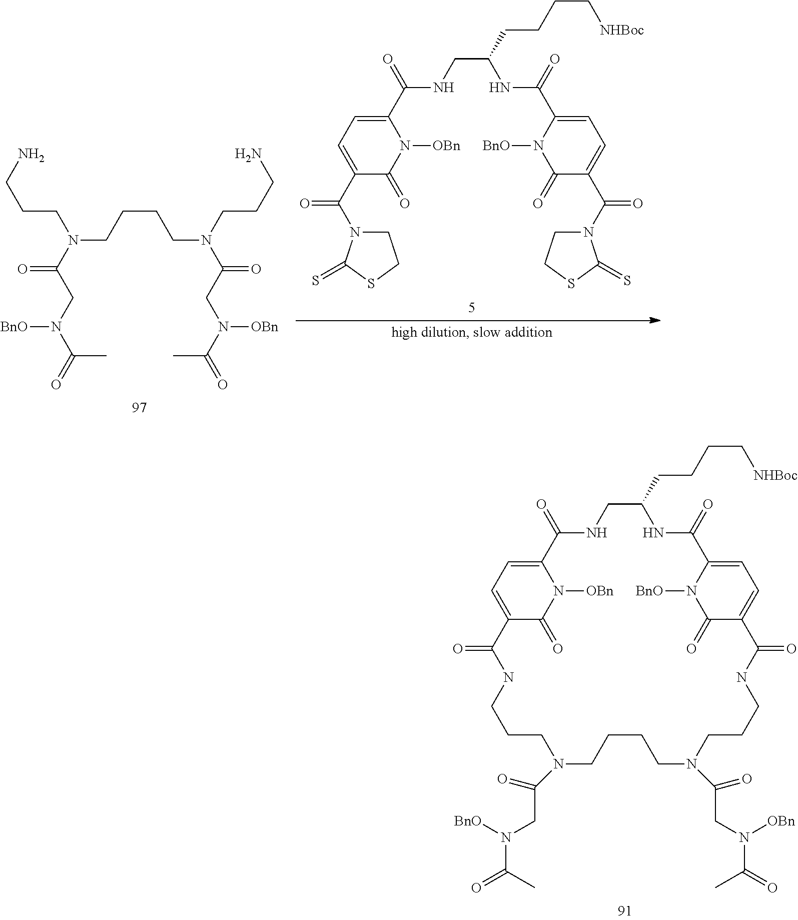

### Example 16. Alternative Synthesis of Protected Bifunctional Chelator 91 (Scheme 16)

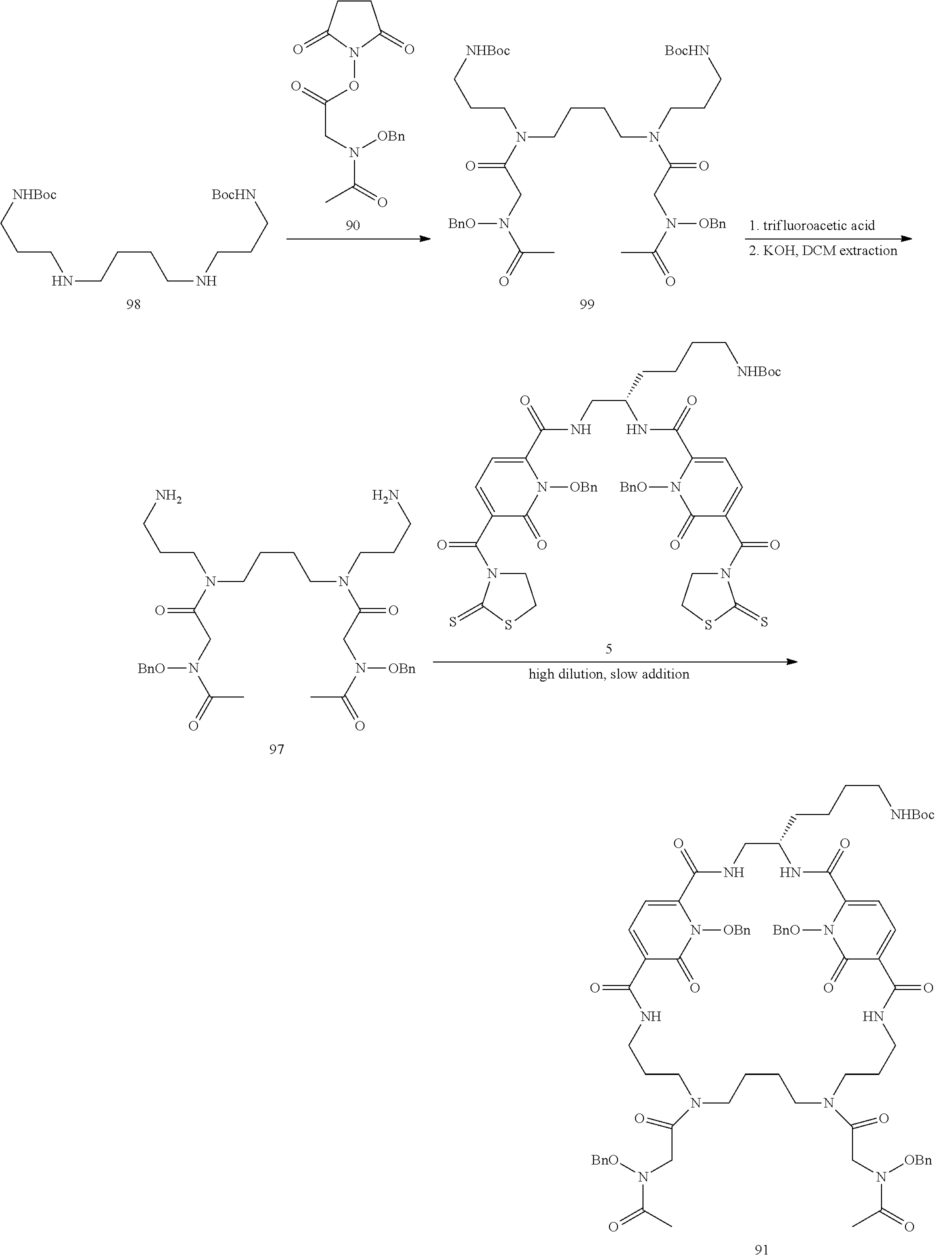

There are at least two examples of generating the carbamate protected spermine 98 reported in the literature. In the one report, spermine is first treated with trifluoroacetic anhydride and DMAP to protect all four nitrogens, reaction of the terminal nitrogen atoms with Boc₂O, and then removal of the TFA protecting groups by treatment with sodium hydroxide in methanol (Perche, P Kotera M, Remy, J-S. MMT, Npeoc-protected spermine, a valuable synthon for the solid phasesynthesis of oligonucleotide oligospermine conjugates via guanidine linkers Bioorg. Med. Chem. 2011, 19, 1972). In another report, spermine is reacted with N-(tert-butoxycarbonyloxy)succinimide (CAS: 13139-12-3) in dichloromethane at 40° C. (Adamczyk, M Fishpaugh J, Heuser K. Chemoselective synthesis of protected polyamines and facile synthesis of polyamine derivatives using O-alkyl-O′—(N-succinimidyl) carbonates, Organic Preparations and Procedures International, 1998, 30:3, 339).

### Example 17. Synthesis of a Mono-Macrocyclic Bifunctional Chelator (Scheme 17)

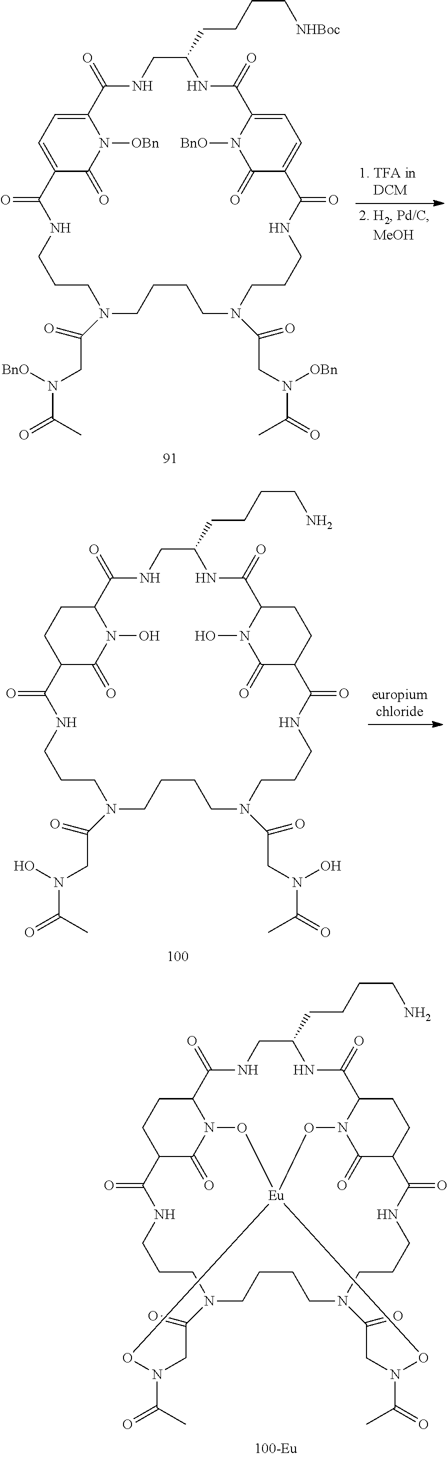

### Example 18. Synthesis of a Mono-Macrocyclic Bifunctional Chelator (Scheme 18)

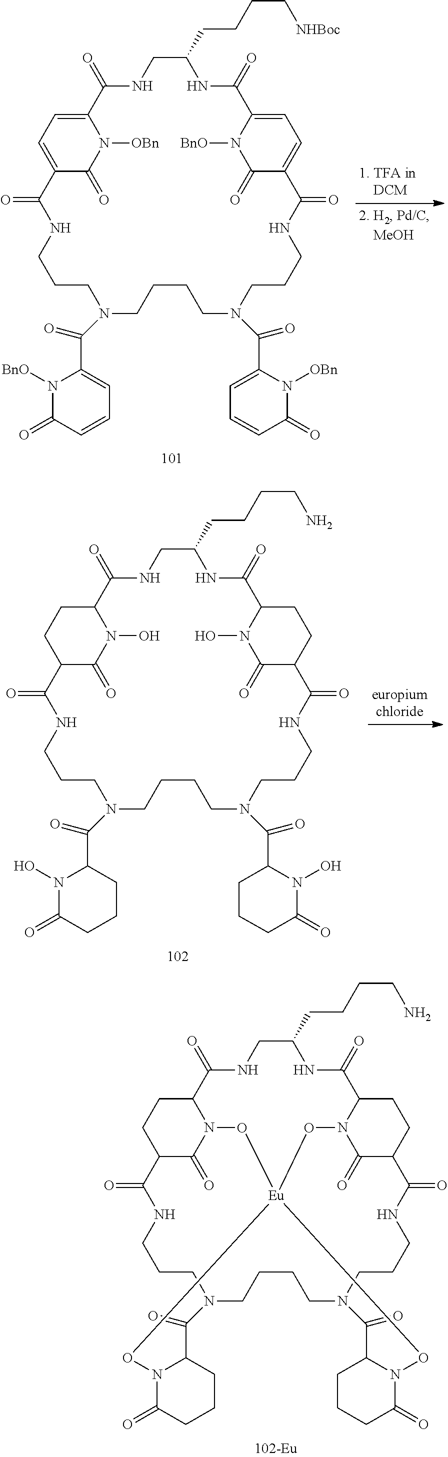

Compound 101 is prepared as reported previously (Tatum D, Xu J, Magda, D, Butlin N. Macrocyclic ligands with pendant chelating moieties and complexes thereof, WO/2019/173639).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A metal chelating agent according to Formula I: 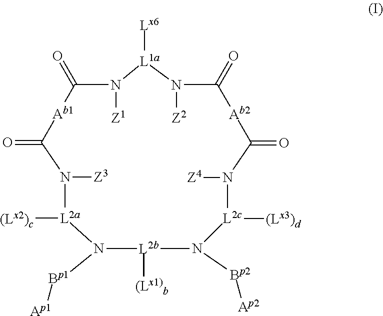
wherein
L^{1a}, L^{2a}, L^{2b}, and L^{2c} are independently selected substituted and unsubstituted C₂-C₆ alkyl;
L^{x1}, L^{x2}, L^{x3}, L^{x4}, L^{x6}, Z¹, Z², Z³ and Z⁴ are independently selected from H, substituted or unsubstituted alkyl and substituted or unsubstituted heteroalkyl, or a linker to a reactive functional group or a targeting moiety, the linker selected from a C₁-C₂₀ substituted or unsubstituted alkyl moiety, and a 1-20 atom length substituted or unsubstituted heteroalkyl moiety;
a, b, c, and d are independently selected from 0 and 1;
A^{b1} and A^{b2} are independently selected from:
(i) substituted and unsubstituted, saturated and unsaturated alkyl selected from straight-chain, branched chain, and cycloalkyl;
(ii) substituted and unsubstituted, saturated and unsaturated heterocycloalkyl; and
(ii) substituted and unsubstituted heteroaryl,
each comprising an oxo and a NOH moiety;
B^{p1} and B^{p2} branching group is selected from:
(i) a bond
(ii) C₁-C₃ comprising an oxo group, and
(iii) —C(O)—;
A^{p1} and A^{p2} are independently selected from:
(a) C₁-C₆-substituted alkyl substituted with a member selected from:
(i) substituted and unsubstituted, saturated and unsaturated cycloalkyl; and
(ii) substituted and unsubstituted, saturated and unsaturated heterocycloalkyl, each comprising an oxo and a NOH moiety; and
(b) (CH₂)ₓC(O)NR¹NR²R³, (CH₂)ₓN(OH)C(O)R¹ and (CH₂)ₓC(O)N(OH)R¹,
wherein R¹, R², and R³ are independently selected from H and C₁-C₆ substituted and unsubstituted alkyl; and
and x is integer from 1-3.

2. The metal chelating agent according to claim 1, wherein A^{b1} and A^{b2} are independently selected from: 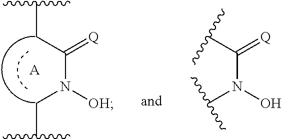
in which ring A is a four-, five-, six-, or seven-member ring, and has at least one degree of unsaturation or is saturated; and Q is independently selected from O, SH, and S.

3. The metal chelating agent according to claim 2, wherein ring A is aromatic.

4. The metal chelating agent according to claim 1 wherein A^{b1} and A^{b2} are independently selected from: 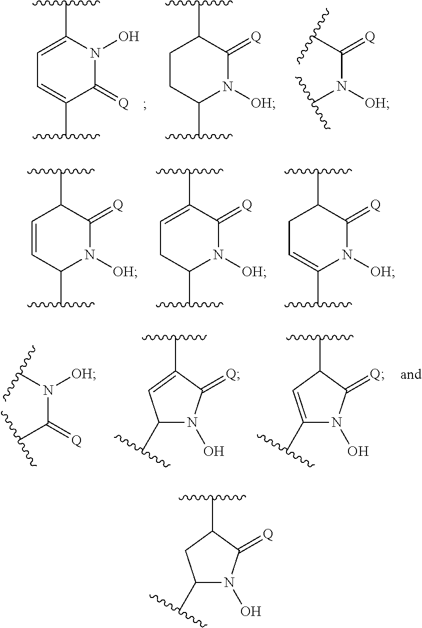
wherein one or more of the positions occupied by H in the formulae above may be substituted with C₁-C₄ alkyl or heteroalkyl, or linker to reactive functional group or targeting moiety.

5. The metal chelating agent according to claim 1, having a structure according to Formula II:

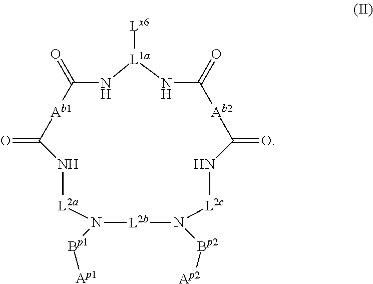

6. The metal chelating agent according to claim 1 according to Formulae III and IV: 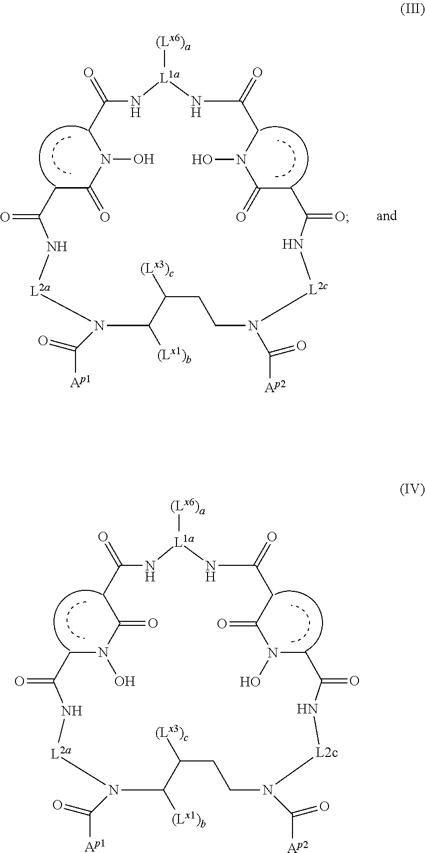
wherein
L^{x1}, L^{x3} and L^{x6} are each independently selected from H, and a bond or a linker to a reactive functional group or a targeting moiety, the linker selected from a C₁-C₂₀ substituted or unsubstituted alkyl moiety, and a 1-20 atom length substituted or unsubstituted heteroalkyl moiety; and
a, b, and c are independently selected from 0 and 1 with the proviso a, b, and c are not all 1, or not more than one or not more than 2 of a, b, and c is 1.

7. The metal chelating agent according to claim 1 wherein A^{p1} and A^{p2} are independently selected from a moiety comprising: 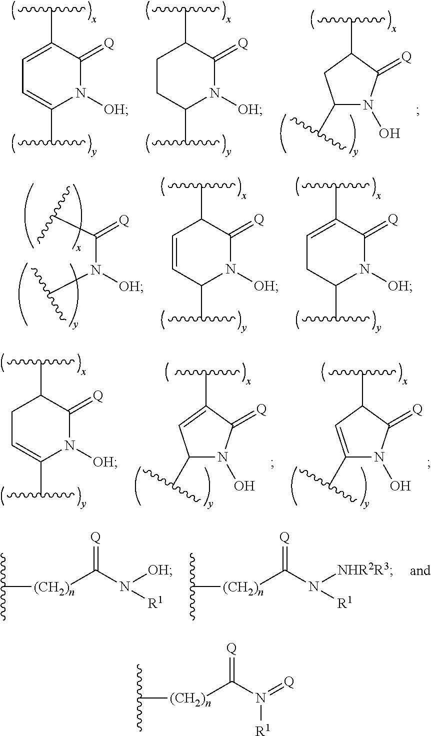
wherein
x and y are 0 or 1 with the proviso that x and y are not both 0 or both 1; n is an integer from 1 to 6; and wherein one or more of the positions occupied by H in the formulae above may be substituted with C₁-C₄ alkyl or heteroalkyl, or linker to reactive functional group or targeting moiety.

8. The metal chelating agent according to claim 1 according to Formulae V or VI:

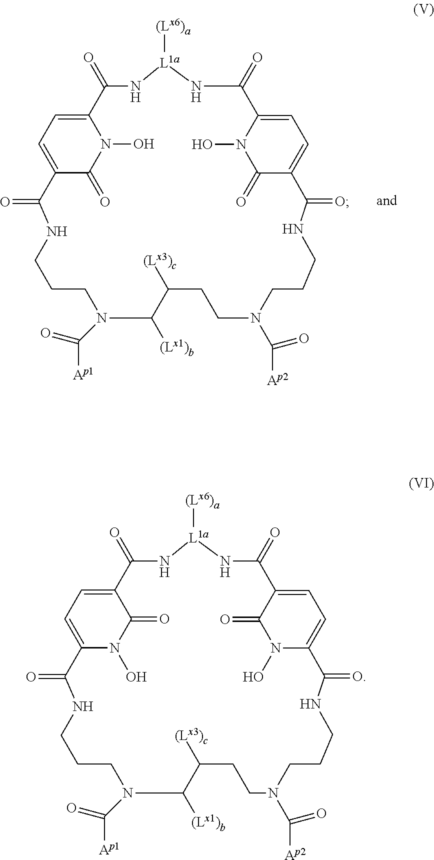

9. The metal chelating agent according to claim 1 in which the metal ion is chelated by the agent through 4, 5, 6, 7, or 8 bonds, each between the metal and an oxygen atom.

10. The metal chelating agent according to claim 1 in which the metal ion is chelated by the agent through at least four bonds, each to a N—O⁻ moiety oxygen atom.

11. The metal chelate according to claim 9, wherein the metal is a radionuclide.

12. The metal chelate according to claim 9, wherein the metal is a Lanthanide or an Actinide.

13. The metal chelate according to claim 9, wherein the metal is selected from Th(IV), Zr(IV), Y(III), Ac(III), Lu(III), Tb(III), In(III), Sc(III), Ga(III), Pb(II), Pb(IV), and Eu(III).

14. The metal chelate according to claim 9, wherein the metal is selected from Th-227, Zr-89, Lu-177, Tb-161, Y-90, Ac-225, In-111, Sc-43, Sc-44, Sc-47, Ga-68, Pb-212.

15. A pharmaceutical formulation comprising the metal chelate of claim 9, and a pharmaceutically acceptable carrier.
